(19) United States
(12) Patent Application Publication
(10) Pub. No.: US 2026/0198970 A1
(43) Pub. Date: Jul. 16, 2026

(54) Systems and Methods for Segmental Three-Dimensional Correction of Spinal Deformities

(71) Applicant: Hazem Bayoumi Elsebaie, Bayonne, NJ (US)

(72) Inventors: Hazem Bayoumi Elsebaie, Bayonne, NJ (US); Behrooz Akbar Akbarnia, San Diego, CA (US); Gregory Michael Mundis, JR., Encinitas, CA (US)

(21) Appl. No.: 19/138,327

(22) PCT Filed: Aug. 15, 2023
(86) PCT No.: PCT/EG2023/000014

§ 371 Date: Jun. 12, 2025

(30) Foreign Application Priority Data
Dec. 22, 2022 (EG) 2022121887

(51) Int. Cl. A61B 17/70; A61B 17/00; A61B 17/56; A61B 34/30; A61B 90/00

(52) U.S. Cl. CPC A61B 17/7023; A61B 34/30; A61B 2017/00075; A61B 2017/00398; A61B 2017/00477; A61B 2017/564; A61B 2090/066

(57) ABSTRACT

Provided are systems and methods to segmentally correct spinal deformities in three dimensions by correcting individual vertebral deviations. A motion control coordinate system calculates individual deviations within the spinal deformity in six degrees of freedom: three linear along the X, Y, and Z axes and three rotational around those axes. The systems can correct any of twelve directions six rotations (three clockwise and three anticlockwise) and six linear (three positive and three negative). Coupler units useful with the system include a coupler body maintaining first and second arms that attach to anchors secured to vertebrae, and a single or multiple joints designed to be selectively manipulated in calculated kinematic solutions. The motion of the joint(s) enable the first and second arms, and hence the attached vertebrae, to move relative to one another from their actual deviated position to a target position to correct the spinal deformity.

### CROSS-REFERENCE TO RELATED APPLICATIONS

This application is the United States national phase of International Patent Application No. PCT/EG2023/000014 filed Aug. 15, 2023, and claims priority to Egyptian Patent Application No. 2022121887 filed Dec. 22, 2022, the disclosures of which are hereby incorporated by reference in their entireties.

### BACKGROUND OF THE INVENTION

### Field of the Invention

The present disclosure is directed to correction of spinal deformities. More particularly, it relates to systems and methods for accurately correcting vertebral deviations in all directions.

### Description of Related Art

Spinal deformities including scoliosis, kyphosis, kyphoscoliosis, spondylolisthesis, etc., pose great challenges in their surgical treatment. These deformities can affect all age groups: young children with early onset scoliosis; adolescent and adults with scoliosis, kyphosis and lytic spondylolisthesis; and adults suffering from degenerative spinal deformities and degenerative spondylolisthesis. Surgical treatment with correction and fixation of the vertebrae remains the mainstay of management for different types of spinal deformities in various age groups. The safety, efficacy and ease of use have been the most important parameters in evaluating the methods and techniques used for correction and instrumented fixation of spinal deformities. Research and development worldwide have always focused on improvement in these fields.

Current spinal fixation and correction systems consist mainly of screws and hooks that are threaded into or attached to the vertebral bodies either posteriorly through the pedicles or anteriorly in the vertebral bodies to act as anchors. The screws are connected by solid rigid rods, either directly in top-loading systems or through side links as in side-loading systems. These screws/anchors are used to try to manipulate the vertebrae and hence the deformed part of the spine into the desired position, then the screw-rod junctions are tightened to fix the spine in the corrected position. However, the motion of the screws and hence the vertebrae is significantly hindered by the fact that the screws are attached to a rigid rod connecting them together. The rigidity of the metal rod does not allow its segmental bending or rotation at each intervertebral level where the intervertebral deformities are occurring. In addition, the rods are usually bent in a global way and mainly in the sagittal plane which can only partially control kyphosis and lordosis. The ability of the screws to rotate and mobilize the vertebrae is very limited by their attachments to the rigid rod. There is no current system capable of true segmental three dimensional (3D) correction of spinal deformities by correcting the individual vertebral deviations at the intervertebral levels where the deformity happens.

The current systems using rigid metallic rods with screws (and hooks) in correction and fixation of spinal deformities have many technical problems concerning their safety, efficacy, ease of use and their inherent inability to do a true segmental 3D correction of spinal deformities. The same traditional basic concepts used in the correction of spinal deformities has not changed for decades due to the limitation posed using rigid rods which is clearly preventing the incorporation new emerging technologies.

One drawback with current systems stems from the observation that connecting a rigid rod to a deformed spine usually requires a variable, non-calculated amount of force. In addition, the force applied to maneuver the rod by rotating it after attaching it the spine increase these forces significantly. The applied forces result in considerable amounts of stress mainly at the implant bone interface. These stresses are unpredictable and unmeasurable; and can result in implant failure, screw loosening, and/or screw displacement with the resultant possible neurological injuries. To overcome this major disadvantage there has been a mushrooming development of tools and instrumentations to connect the pedicle screws to the pre-bent rods in an easier, gradual and least risky ways including poly-axial screws, reduction screws, poly-axial bolts, persuaders, dual locking nuts, etc. Unfortunately, nothing as of yet has been fully satisfactory.

Another concern with traditional systems is that there is no true segmental control of each vertebra at the intervertebral levels where the deformity is occurring; any correction achieved with these systems is rather partial and global in nature. Individual vertebral deviations cannot be corrected by any of these traditional concepts and systems, therefore using these tools will not achieve true full segmental three-dimensional correction spine deformities. None of these systems offers control on the individual vertebrae in all axes of rotation and the attachment of the screws to the rigid rod prevent free independent vertebral mobility.

An additional problem with existing systems is that once the rod is connected to the screws and tightened, the whole system is locked in; most of its motions and further significant adjustments are limited and can be risky. In-situ bending techniques were adopted to overcome this limitation, however they did not achieve the required adjustments and, in many times, proved risky with a possibility of screws backing out.

A further concern raised by existing systems is that partial correction achieved with these systems cannot be calculated in angles or distances. Therefore, the accuracy of such correction cannot be verified or controlled therefore preventing any adoption of new technologies that require high accuracy instruments and implants.

### SUMMARY OF THE INVENTION

The inventors of the present disclosure has recognized a need to address one or more of the above-mentioned problems.

With the current advancements in imaging techniques, radiological 3D measurements and 3D printing; in addition to the improvements in medical technology, navigation, robotics, computer aided diagnosis (CAD) and artificial intelligence; there is a need for high-accuracy surgical techniques and implants. Aspects of the present disclosure entail changes to the traditional surgical pathways and concepts for the treatment of spinal deformities; in some embodiments, systems of the present disclosure are capable of incorporating these new, emerging technologies.

Some methods and systems of the present disclosure are based on identifying each vertebra within the spinal deformity as an object that follows a Motion Control Coordinate System in free space. In free space, an object is considered to have six degrees of freedom: three linear, along the X, Y, and Z-axes and three rotational around those axes; all motions are either translations along or rotations about the coordinate axes. A spinal deformity is formed of multiple segmental deformities occurring mainly at the intervertebral levels (at the level of the discs and facet joints). Each intervertebral motion segment includes two adjacent vertebrae and an intervertebral disc in-between. The vertebrae within the curve are deviated from its normal anatomical position to an abnormal deviated position. The abnormal deviated position of the vertebra can be determined or calculated in different directions and by correcting these deviations, the vertebrae should revert to their normal anatomical position.

With some embodiments of the present disclosure, a spinal deformity is, or can be, digitized by determining or calculating the individual vertebral deviations of all the vertebrae involved within the deformed segment. As a point of reference, digitization is the process of converting physical information into a digital format (numbers); through this process the information perceived from the patient's radiograph is converted to a set of numerical data that we used to design systems and implants capable for the first time to achieve a true 3D segmental correction of the deformity.

A normal human vertebra in a non-deformed spine has zero axial rotation, zero coronal angulation and follow the normal sagittal dorsal kyphosis and lumbar lordosis in the sagittal plane. A spinal deformity is the sum of multiple intervertebral segmental deformities resulting from individual vertebral deviations. Each intervertebral motion segment includes 2 adjacent vertebrae and an intervertebral disc in-between. Scoliosis, the most common spinal deformity, is a three-dimensional (3D) deformity that can be associated with lateral deviation in the coronal plane, thoracic hypokyphosis in the sagittal plane and rotation in the axial plane. CT images, biplanar radiographic 3D images and 3D printing are currently utilized to optimize preoperative, intraoperative, and postoperative care of patients with spine deformity in addition to their use in custom or patient specific implants. These new tools will be used for an accurate assessment and measurement of 3D vertebral deviations occurring at each intervertebral motion segment. By measuring the local coordinates of each vertebra, some systems and methods of the present disclosure can determine or calculate the three-dimensional deviations of this individual vertebra. Each segmental intervertebral deformity can be determined or calculated as deviations of one vertebra in relation to the adjacent one by measuring the differences between the coordinates of the two adjacent vertebrae. By determining or calculating the rotational and linear deviations, some systems and methods of the present disclosure can identify the exact distances and angles of deviations of the vertebra. By calculating the rotational and linear deviations, one can digitize the deformity (e.g., converting physical information that is seen on radiographs into a digital format using numbers) in some embodiments. Regardless, identifying and correcting deformities can result in true full 3D correction of each intervertebral deformity and consequently the whole spinal deformity.

Regardless of how a particular spinal deformity is assessed or characterized (e.g., based on the digitized methodologies mentions above in some non-limiting examples), some systems of the present disclosure incorporate features configured to utilize or use these assessments (e.g., numbers) to accurately correct the vertebral deviation(s) in question, align the deformity and fix the spine in the corrected position. For examples, some systems of the present disclosure are directed to a correction unit. In some embodiments, the single unit of the system includes a correction-fixation coupler with a first and second arms extending from both ends. The arms attach proximally and distally to first and second anchors that are secured to a first and second vertebrae through the pedicle posteriorly or directly in the vertebral body anteriorly. Each coupler can include within its body a single or multiple uniplanar/uniaxial joints each of which controls a specific linear or rotational motion. The number and type of the joints included in each coupler will be guided by the number and type of linear and/or rotary deviations each coupler is intended to correct. Each joint can allow the rotary motion in clockwise and anti-clockwise directions, and the linear motion in positive and negative directions. Each uniplanar/uniaxial joint in the coupler can be selectively mobilized to predetermined angle in rotation or distance in linear motion to collectively achieve the target position. The joint can be selectively mobilized manually with a driver, automatically via an external motor, or remotely via a controller and internal motor within the body of the coupler to achieve the desired end-effector motion. The joints'function can be motorized and operated by using a remote or switch so that each action can be initiated by the clinician or operator; or can be automated so they are programmed to function via set of commands using a smart hub. Each turn or part of a turn of the driver or the motor is accurately calibrated to mobilize the specific joint in a calculated distance in linear motion or degree in rotational motion; this will result in fully calibrated control of the motion of each joint. The torque/force applied by the driver/motor can optionally be measured by a tool of the present disclosure to determine the amount of stress applied which can be transmitted to the implant-bone interface. The joints within the body of the coupler are in one of its configurations to be self-locking joints which will guarantee that any motion/correction achieved will remain in its new acquired position and will be locked in this position until the joint is actively mobilized according to the need. This will also allow the correction to be done gradually and back and forth alternating not only between the joints within the individual coupler but also between different couplers at different intervertebral levels within the deformity. Once the targeted correction is achieved, the joints in the coupler are fully locked and the correction-fixation coupler itself is used to fix the spine in the corrected position. With these and related embodiments, systems and methods of the present disclosure can provide a full, 3D correction of the deformity in all directions. Another option of the systems of the present disclosure is to use the couplers as a temporary device to correct the vertebral deviations and consequently the spinal deformity and then the couplers are removed after the spine is fixed with a second segmental fixation apparatus or a contoured conventional solid rod.

### BRIEF DESCRIPTION OF THE DRAWINGS

FIG. 1 is a perspective view of a human body and identifying a coordinate system and anatomical body planes;

FIG. 2 is a perspective view of a vertebral segment and identifying a vertebral motion coordinate system with six degrees of freedom and twelve directions of motion;

FIG. 3 is a top view of the vertebral segment of FIG. 2 and illustrating axial rotation of a first vertebra in relation to a second vertebra;

FIG. 4 is a posterior view of a vertebral segment of FIG. 2 and illustrating coronal tilt of a first vertebra in relation to a second vertebra;

FIG. 5 is a lateral or side view of the vertebral segment of FIG. 2 and illustrating sagittal angulation a first vertebra in relation to a second vertebra;

FIG. 6 is a lateral view of the vertebral segment of FIG. 2 and illustrating showing cranio-caudal linear displacement (compression-distraction);

FIG. 7 is a side view of the vertebral segment of FIG. 2 and illustrating antero-posterior translation;

FIG. 8 is a posterior view of the vertebral segment of FIG. 2 and illustrating medio-lateral translation;

FIG. 9 is a perspective view of a correction-fixation coupler unit in accordance with principles of the present disclosure;

FIG. 10 is a top view of the correction-fixation coupler unit of FIG. 9;

FIG. 11 is a side view of the correction-fixation coupler unit of FIG. 9;

FIG. 12 is an end view of the correction-fixation coupler unit of FIG. 9;

FIG. 13 is an opposite end view of the correction-fixation coupler unit of FIG. 9;

FIG. 14 is an exploded, perspective view of the correction-fixation coupler unit of FIG. 9;

FIG. 15 is a top view of the correction-fixation coupler unit of FIG. 9 and generally identifying a joint mechanism type A;

FIG. 16 is an enlarged cross-sectional view of the correction-fixation coupler unit of FIG. 15, taken along the line A-A;

FIG. 17 is an exploded, perspective view of a portion of the correction-fixation coupler unit of FIG. 9 including components of the joint mechanism type A of FIG. 15;

FIG. 18 is a perspective view of components of the joint mechanism type A of FIG. 17 upon final assembly;

FIG. 19 is a top view of the correction-fixation coupler unit of FIG. 9 and generally identifying a joint mechanism type B;

FIG. 20 is an enlarged cross-sectional view of the correction-fixation coupler unit of FIG. 19, taken along the line B-B;

FIG. 21 is an exploded, perspective view of a portion of the correction-fixation coupler unit of FIG. 9 including components of the joint mechanism type B of FIG. 19;

FIG. 22 is a perspective view of components of the joint mechanism type B of FIG. 19 upon final assembly;

FIG. 23 is a side view of the correction-fixation coupler unit of FIG. 9 and generally identifying a joint mechanism type C;

FIG. 24 is an enlarged cross-sectional view of the correction-fixation coupler unit of FIG. 23, taken along the line C-C;

FIG. 25 is an exploded, perspective view of a portion of the correction-fixation coupler unit of FIG. 9 including components of the joint mechanism type C of FIG. 23;

FIG. 26 is a perspective view of components of the joint mechanism type C of FIG. 23 upon final assembly;

FIG. 27 is a lateral view of a deformed vertebral motion segment with both vertebrae sagitally angulated in relation to each other along with the correction-fixation coupler unit of FIG. 9 attached to both vertebrae;

FIG. 28 is a lateral view of the vertebral motion segment of FIG. 27 following correction of the sagittal angulation by the correction-fixation coupler unit;

FIG. 29 is a top view of a deformed vertebral motion segment with both vertebrae axially rotated in relation to each other along with two of the correction-fixation coupler units of FIG. 9 attached to both vertebrae;

FIG. 30 is a top view of the vertebral motion segment of FIG. 29 following correction of the axial rotation by the correction-fixation coupler unit;

FIG. 31 is a posterior view of a deformed vertebral motion segment with both vertebrae axially rotated in relation to each other along with two of the correction-fixation coupler units of FIG. 9 attached to both vertebrae;

FIG. 32 is a posterior view of the vertebral motion segment of FIG. 31 following correction of the axial rotation by the correction-fixation coupler units;

FIG. 33 is a posterior view of a deformed vertebral motion segment with both vertebrae coronally tilted in relation to each other along with two of the correction-fixation coupler units of FIG. 9 attached to both vertebrae;

FIG. 34 is a posterior view of the vertebral motion segment of FIG. 33 following correction of the coronal tilt by the correction-fixation coupler units;

FIG. 35 is a top view of an embodiment of a correction-fixation coupler unit in accordance with principles of the present disclosure including three joint mechanisms and useful for correcting scoliosis-caused spinal deformities;

FIG. 36 is a lateral view of a vertebral motion segment and the correction-fixation coupler unit of FIG. 35 attached to vertebral screws inserted in the lateral side of the vertebral bodies;

FIG. 37 is an anterior view of a vertebral motion segment and the correction-fixation coupler unit of FIG. 35 attached to vertebral screws inserted in the lateral side of the vertebral bodies;

FIG. 38 is a lateral view of a vertebral motion segment showing a first vertebra anteriorly translated on a second vertebra, and a correction-fixation coupler unit in accordance with principles of the present disclosure attached to the two vertebrae;

FIG. 39 is a top view of another correction-fixation coupler unit in accordance principles of the present disclosure and including a joint mechanism type A and a joint mechanism type B;

FIG. 40 is a top view of another correction-fixation coupler unit in accordance principles of the present disclosure and including a joint mechanism type B and a joint mechanism type C;

FIG. 41 is a top view of another correction-fixation coupler unit in accordance principles of the present disclosure and including a joint mechanism type A and a joint mechanism type C;

FIG. 42 is a top view of another correction-fixation coupler unit in accordance principles of the present disclosure and including a joint mechanism type A;

FIG. 43 is a top view of another correction-fixation coupler unit in accordance principles of the present disclosure and including a joint mechanism type B;

FIG. 44 is a top view of another correction-fixation coupler unit in accordance principles of the present disclosure and including a joint mechanism type C;

FIG. 45 is a top view of another correction-fixation coupler unit in accordance principles of the present disclosure and including a joint mechanism type D;

FIG. 46 is a side view of another correction-fixation coupler unit in accordance principles of the present disclosure and including a joint mechanism type E;

FIG. 47 is a posterior view of a vertebral column and an arrangement of correction-fixation coupler units in accordance with principles of the present disclosure inserted at each single intervertebral level bilaterally without skipping any vertebra;

FIG. 48 is a posterior view of a vertebral column and an arrangement of correction-fixation coupler units in accordance with principles of the present disclosure inserted posteriorly at every other level in a symmetrically bilateral arrangement;

FIG. 49 is a posterior view of a vertebral column and an arrangement of correction-fixation coupler units in accordance with principles of the present disclosure inserted posteriorly at every other level in non-symmetrical bilateral alternating arrangement;

FIG. 50 is a posterior view of two vertebral motion segments exhibiting coronal tilt along four correction-fixation coupler units in accordance with principles of the present disclosure coupled to three vertebrae; and

FIGS. 51-56 illustrate a method of deploying a correction-fixation coupler unit and a fixation apparatus to correct a spinal deformity in accordance with principles of the present disclosure.

### DESCRIPTION OF THE INVENTION

Some aspects of the present disclosure are directed to a spine correction and fixation unit and systems incorporating spine correction and fixation units for treatment of spinal deformities. The disclosure further provides methods for measuring or evaluating vertebral deviations within any type of spinal deformity and using these measurements or evaluations to choose the appropriate spinal correction and fixation units that will be used to correct each motion segment deformity by correcting individual vertebral deviations. The units, systems, and methods of the present disclosure may be used to treat spinal deformities including but not limited to scoliosis, kyphosis, kyphoscoliosis, degenerative scoliosis, spondylolisthesis, etc. In some embodiments, the correction and fixation units, and systems comprising such units, are configured to correct the individual vertebral deviations thereby correcting and fixing the spine deformity. The units and collectively the system of the present disclosure can, for example, correct axial rotation, coronal tilt, sagittal angulation, compression-distraction, medio-lateral and antero-posterior translations; therefore, the systems and methods of the present disclosure can correct any vertebral deviation in all 6 axes of motion. The methods and systems of the present disclosure are, in some non-limiting embodiments, designed in such a way that adapts the latest evolving technologies by using only numbers (angles in rotation and distances in linear motions) to analyze all types of vertebral deviations; in addition, some embodiments of the present disclosure incorporate uniaxial/uniplanar (robotic) joints to allow those of ordinary skill in the art make the function and configuration of such units and systems motorized and automated.

The following embodiments are described in the context of a spinal correction and fixation methods and systems used to treat spinal deformities. Some descriptions of the present disclosure and figures utilize common or accepted motion terminology and the familiar human anatomical and spine surgery terminologies. Some examples in accordance with principles of the present disclosure will now be described, by way of example only, in detail with reference to the accompanying figures; not all features of the various applications and implementations of the present disclosure are described in the following figures.

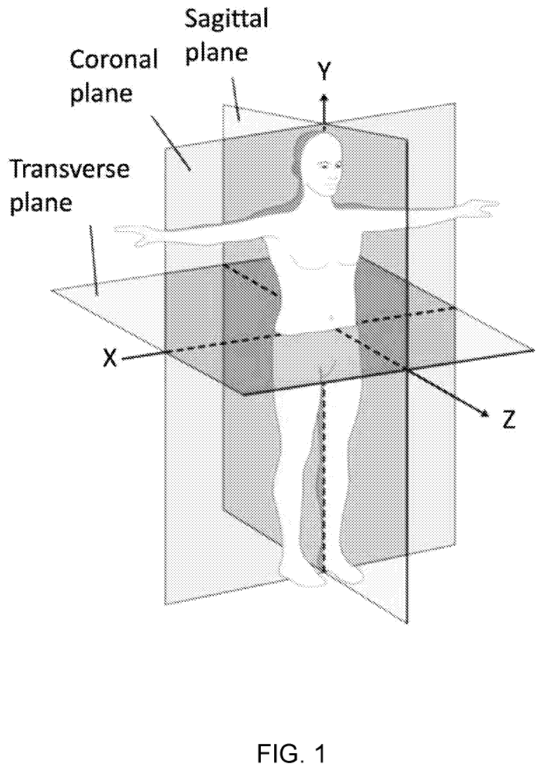

As a point of reference, FIG. 1 shows the anatomical planes of the body in relation to the motion coordinate system. Body planes are hypothetical geometric planes that divide the human body into sections. Mainly these body planes are used in human anatomy to describe the direction and location of body structures. A human body in the anatomical position is described with the help of a coordinate system, which includes three-axis (X, Y, and Z). The X-axis is going from left to right (or medio-lateral), Z-axis from front to back (antero-posterior), and Y-axis from up to down (cranio-caudal). In anatomical terminology, three references plane are considered standard planes; these planes differentiate the body portions. The sagittal (Y-Z) plane separates the body right and left portions. The coronal (Y-X) plane divides the body into anterior and posterior portions. The axial or transverse (X-Z) plane divides the body into cranial and caudal portions.

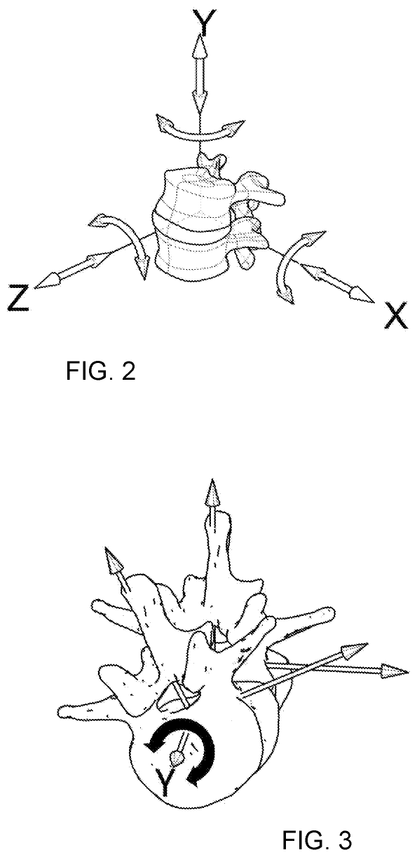

FIG. 2 depicts a vertebral motion coordinate system with six degrees of freedom and twelve directions of motion. In particular, FIG. 2 reflects a vertebral motion segment comprising two vertebrae and an intervertebral disc in-between. The arrows in FIG. 2 indicate the vertebral motion coordinate system with six degrees of freedom. The rotation motion around X/left-right axis/medio-lateral is referred to as “sagittal angulation”, while the linear motion is referred to as “medio-lateral translation”. The rotation motion around Z/anterior-posterior axis is referred to as “coronal tilt”, while the linear motion is referred to as “antero-posterior translation”. The rotation motion around Y/cranio-caudal (or cranial-caudal) axis is referred to as “axial rotation”, while the linear motion is referred to as “compression-distraction”. Each vertebral rotation motion can occur in two directions: clockwise and anticlockwise; each vertebral linear motion can occur in two directions: positive and negative. Therefore the two vertebrae can move in relation to each other in six degrees of freedom in twelve different directions.

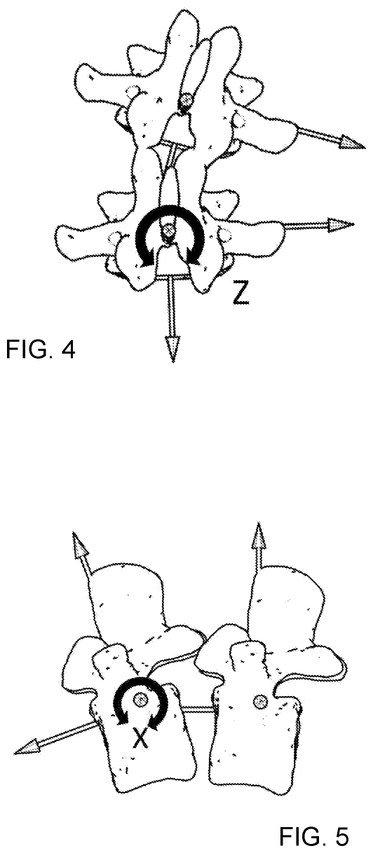

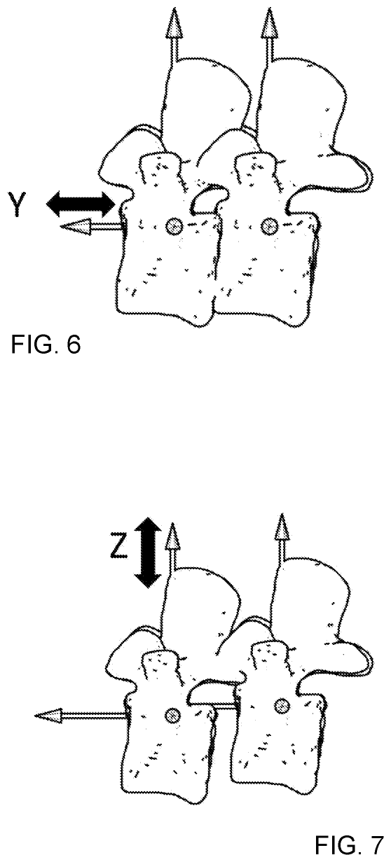

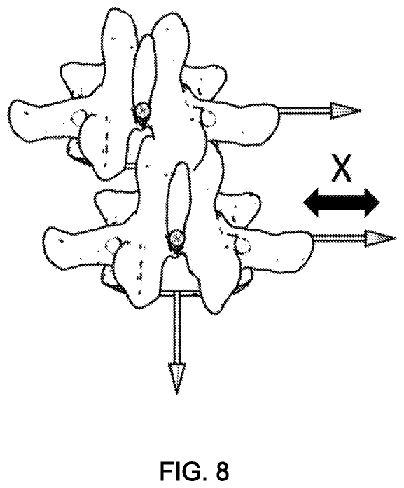

With the above designations in mind, spinal deformities can assume a wide variety of forms. For example, FIG. 3 is a top view of a vertebral motion segment showing axial rotation of one vertebra in relation to the other vertebra. The black arrow in FIG. 3 reflects the clockwise and anticlockwise rotation around the cranio-caudal/Y axis. FIG. 4 is a posterior view of a vertebral motion segment showing coronal tilt of one vertebra in relation to the other vertebra. The black arrow in FIG. 4 reflects the clockwise and anticlockwise rotation around the antero-posterior/Z axis. FIG. 5 is a lateral or side view of a vertebral motion segment showing sagittal angulation of one vertebra in relation to the other vertebra. The black arrow in FIG. 5 reflects the clockwise and anticlockwise rotation around the right-left/X axis. FIG. 6 is a lateral view of a vertebral motion segment showing cranio-caudal linear displacement (compression-distraction) of one vertebra in relation to the other vertebra (narrowing of disc space or compression is shown in this figure). The black arrow in FIG. 6 reflects the positive and negative linear displacement along the cranio-caudal/Y axis. FIG. 7 is a side view of a vertebral motion segment showing antero-posterior translation of one vertebra in relation to the other vertebra (anterior translation or antero-lithesis is shown in FIG. 7). The black arrow in FIG. 7 reflects the positive and negative linear displacement along the antero-posterior/Z axis. FIG. 8 is a posterior view of a vertebral motion segment showing medio-lateral translation of one vertebra in relation to the other vertebra. The black arrow in FIG. 8 reflects the positive and negative linear displacement along the right-left/X axis.

Some systems and methods of the present disclosure use the vertebral motion coordinate system shown in FIG. 2 (with the six degrees of freedom in the possible twelve directions of motion) to evaluate or measure the individual vertebral deviations at each motion segment for a particular patient. These evaluations or measurements can be done at each intervertebral level to determine or calculate one or more parameters, such as: 1) Number: single or multiple deviation/s, and if multiple, how many deviations; 2) Type: around which axis of rotation and which axis of linear displacement; 3) Direction(s): for each rotation motion which direction (clockwise or anticlockwise) and for each linear motion which direction (positive or negative); 4) Amount: for rotation in degrees and for linear displacement in units of measurement (e.g., millimeters). As described in greater detail below, the correction and fixation unit or coupler of the systems of the present disclosure can be attached to two vertebrae to correct the vertebral deviation of the first vertebra in relation to the second vertebra and therefore the intervertebral deformity will be corrected. The unit or correction-fixation coupler inserted between the two vertebrae can, in some embodiments, include the number and types of joints capable of selectively correcting each vertebral deviation; the configuration of each joint of the corresponding unit will depend on the direction of deviation and the amount of deviation; all the four parameters identified above are determined or measured in accordance with some methods of the present disclosure. By way of non-limiting example, scoliosis, which is the most common spinal deformity, is a three-dimensional (3D) deformity that can be associated with lateral deviation in the coronal plane, thoracic hypokyphosis in the sagittal plane and rotation in the axial plane. Therefore, the expected individual vertebral deviation within the scoliotic spinal deformity is expected to occur around the three rotation axes: axial rotation around the Y axis, sagittal angulation around the X axis, and coronal tilt around the Z axis.

A wide range of spinal deformities can be corrected by coupler units (or “correction-fixation coupler units) of the present disclosure. The coupler units of the present disclosure can take various forms, and generally provide one or more uniaxial joints. Different embodiments of coupler units in accordance with principles of the present disclosure are described below, each presenting a different number of joints and each well-suited to address or correct designated spinal deformities. In some embodiments, two or more of the coupler units can be provided to a clinician as part of a system of the present disclosure. In general terms, the coupler units of the present disclosure can include one or more of five different types of joint mechanisms capable of correcting deviations in all six planes of motion. The five different joint mechanism types of the present disclosure are designated in the figures and descriptions below as joint A, joint B, joint C, joint D, and joint E. Joint A corrects axial rotation, joint B corrects sagittal angulation, joint C corrects linear compression distraction and using joint C on the right and left side of the vertebral body will correct coronal tilt rotation, joint D corrects mediolateral translation, and joint E corrects antero-posterior translation. The correction fixation coupler units of the present disclosure can be designed in many configurations to be able to correct any type, number, and combination of vertebral deviation occurring in any of the six planes of motions. A correction-fixation coupler unit can contain either a single or multiple joints or joint mechanisms; the choice of the number of uniplanar/uniaxial joint mechanisms in each coupler depends on the number of planes of deviation of the vertebra a particular coupler unit is intended to correct. The choice of the type (A, B, C, D, E) of the joint mechanisms within the particular coupler unit will depend on the planes of linear and/or rotational deviations in which the vertebrae is deviated. Effectively, then, with the systems of the present disclosure, a clinician is provided with the ability to choose a coupler unit best suited for a particular patient. Based on, for example, 3D imaging of the patient's spine, a clinician can decide on the type(s) and number of uniaxial joint(s) s/he needs with the particular coupler unit for a particular spinal level of the patient. For example, the selected coupler unit can include one, two, three, or more joints according to the number and directions of deviation(s) that need correction for the particular patient. According to the vertebral deviation(s) that require correcting, the suitable coupler unit can include one joint type to address one deviation, two or three joints to address two or three deviations, or three or more joints to address three or more deviations. Regardless of exact form, the joints or joint mechanisms of the present disclosure can be “lockable” (e.g., alternate between being unlocked and actively locked by a screw, pin, or the like) or “self-locking” (as described in some embodiments below). With the optional lockable designs, the lockable one degree-of-freedom joints of the present disclosure include, but are not limited to, revolute joints, translational joints, etc., that can be actively locked by a screw, pin or similar means.

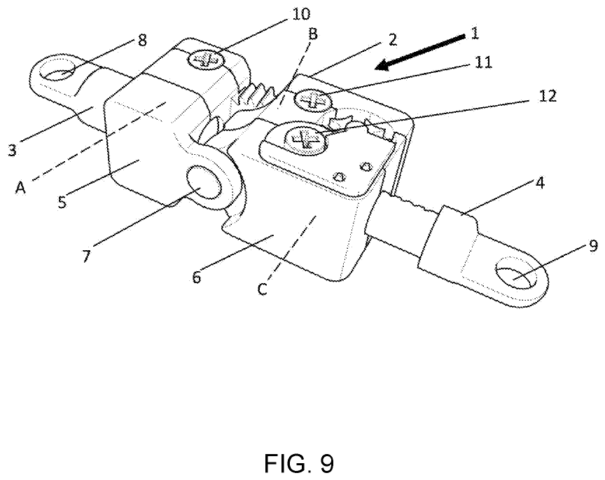

One example of a correction-fixation coupler unit 1 in accordance with principles of the present disclosure is shown in FIG. 9. In some non-limiting examples, the correction units of the present disclosure, such as the unit 1, can include three joint mechanisms for controlling the three types of motion associated with the scoliosis spinal deformities mentioned above. The correction-fixation coupler unit 1 can be used to correct the most common vertebral deviations in scoliosis as it comprises three uniplanar/uniaxial joints controlling the three rotation motions required to correct scoliotic deformity namely axial rotation, sagittal angulation, and coronal tilt. In some examples, then, the coupler unit 1 can be referred to as a “scoliosis correction-fixation coupler” or “scoliosis correction-fixation coupler unit”. The correction-fixation coupler unit 1 comprises a coupler 2, a first arm 3 extending from a first end of the coupler 2, and a second arm 4 extending from an opposing, second end of the coupler 2. The coupler 2 comprises two parts: the first coupler body part 5 and a second coupler body part 6. The coupler body parts 5, 6 are connected around an axis 7. The first arm 3 has an aperture 8 designed to receive and attach to a post of a first anchor (not shown) secured in a first vertebra as described below. The second arm 4 has an aperture 9 designed to receive and attach to a post of a second anchor (not shown) secured in a second vertebra. The coupler 2 further comprises or defines three uniaxial/uniplanar joints (identified in FIG. 9 generally at A, B, and C), each of which allows mobilization of the first arm 3 in relation to the second arm 4 around or along one single specific axis. Each of the three joints A, B, C within the coupler 2 has a separate actuator to mobilize the corresponding joint. In the view of FIG. 9, the first joint A can be operated or articulated by a first actuator 10; the second joint B can be operated or articulated by a second actuator 11; the third joint C can be operated or articulated by a third actuator 12. The actuators 10, 11, 12 can assume various forms, and in some non-limiting examples can be a screw, with a screw head of the screw being available or exposed for interfacing by a clinician or user. With these and related embodiments, the screw head is designed to receive a manual driver or external motor capable of turning the screw head clockwise or anticlockwise in an exact calculated amount. In some embodiments, each of the actuators 10, 11, 12 is calibrated in a way that turning the corresponding screw head in a calculated amount in a certain direction mobilizes the corresponding joint for a calculated degree in rotational joint or calculated distance in linear joint.

From these descriptions, then, the body of the coupler 2 comprises the three joints A, B, C operated by the three actuators (e.g., screw heads) 10, 11, 12 respectively. The first actuator (e.g., screw head) 10 controls the first joint A embedded in the first coupler body part 5; turning the first actuator/screw head 10 will operate the first joint A which in turn will mobilize the first arm 3 to rotate around its own axis. The second actuator (e.g., screw head) 11 controls the second joint B present between first coupler body part 5 and the second coupler body part 6, and is partially embedded in the second coupler body part 6; turning the second actuator/screw head 11 will operate the second joint B which in turn will mobilize the first coupler body part 5 and the second coupler body part 6 in relation to each other around a common axis 7. The third actuator (e.g., Screw head) 12 controls the third joint C embedded in the second coupler body part 6; turning the third actuator/screw head 12 will operate the third joint C which in turn will mobilize the second arm 4 to elongate and shorten by its linear motion along its own axis moving in and out from the second coupler body part 6.

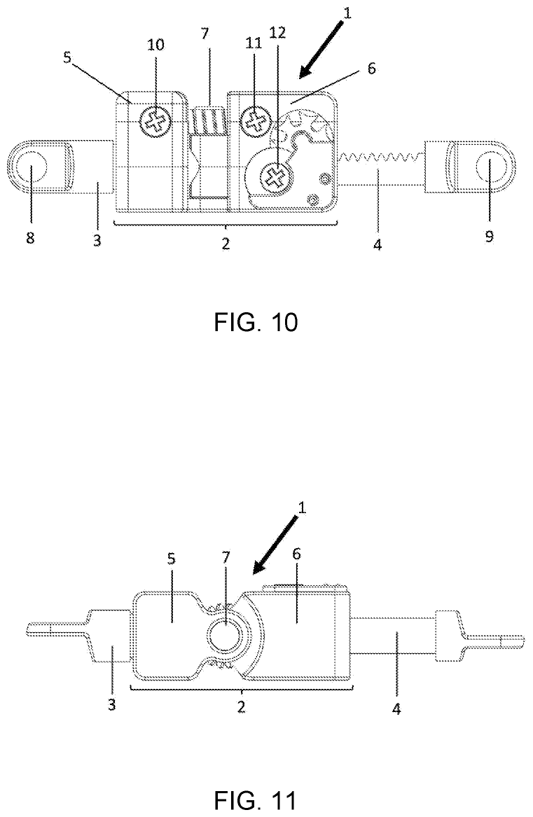

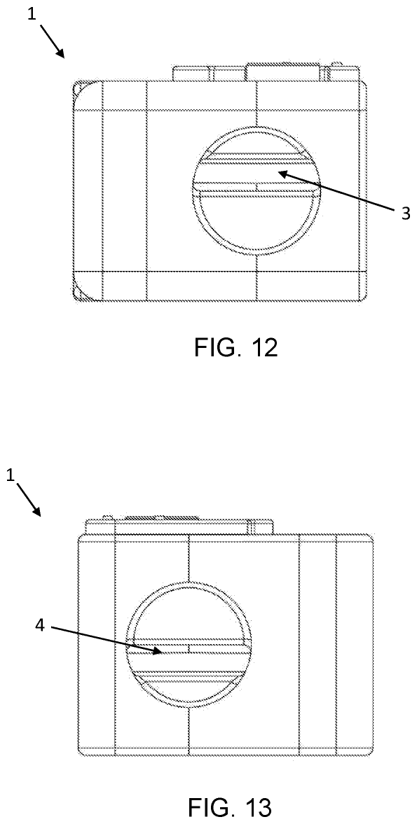

Top and side views, respectively, of the correction-fixation coupler unit 1 are provided in FIGS. 10 and 11. Once again, the correction-fixation coupler unit 1 comprises a the coupler 2, the first arm 3 extending from the first end of the coupler 2, and the second arm 4 extending from the second end of the coupler 2. The body of the coupler 2 comprises two parts: the first coupler body part 5 and the second coupler body part 6. The coupler body parts 5, 6 are connected around the axis 7. End views of the coupler unit 1 are shown in FIGS. 12 and 13.

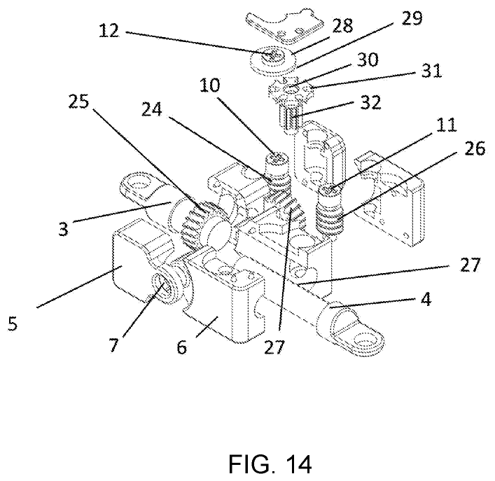

The various joint(s) provided by the coupler units of the present disclosure, for example the coupler unit 1, can take various forms. Some non-limiting examples reflected by the exploded view of the coupler unit 1 in FIG. 14. The body of the coupler 2 (FIG. 10) is open showing the three joint mechanisms, as well as the first arm 3 and the second arm 4. In some embodiments, the first joint A (identified in FIG. 9) can be a friction based locking joint and can includes a worm or worm-screw 24 (e.g., the driver) with the screw head 10, and a worm-wheel 25 (e.g., the driven) which is part of the first arm 3 that is otherwise embedded in the first coupler body part 5. When a manual screwdriver or a motor is used to turn the worm-screw head 10 it will turn the worm 24 which subsequently turn the first arm 3 (otherwise carrying the worm-wheel 25), causing the first arm 3 to rotate around its own axis. In some embodiments, the non-limiting example worm gear self-locking mechanism is based on the concept that the worm can drive the gear or wheel, but due to the inherent friction the gear cannot turn (back-drive) the worm.

The second joint B (identified in FIG. 9) can be a friction based locking joint in some non-limiting examples, and includes a worm or worm-screw 26 (e.g., the driver) with the screw head 11, and a worm wheel 27 (e.g., the driven) which is part of the first coupler body part 5. When a manual screwdriver or a motor is used to turn the worm-screw head 11 it will turn the worm wheel 27 with the first coupler body part 5; as a result, the first couple body part 5 will rotate in relation to the second coupler body part 6 around the axis 7.

The third joint C (identified in FIG. 9) can by a mechanical locking joint in some non-limiting examples, such as a cam-based locking “Geneva” mechanism, and can include a crank 28 which carries a roller or a knob 29, and a Geneva wheel 30 with multiple slots 31. The crank 28, which usually rotates by turning the screw head 12, carries a roller (hidden) to engage with the slots 31. During one revolution of the crank 28, the Geneva wheel 30 rotates a fractional part of the revolution, the amount of which is dependent upon the number of the slots 31. The circular segment attached to the crank 28 effectively locks the wheel 30 against rotation when the roller is not in engagement and positions the wheel 30 for correct engagement of the roller 29 with the circumferentially-next slot 31. The Geneva wheel 30 carries a pinion 32 which effectively engages a rack 27 which is part of the second arm 4. When a manual screwdriver or a motor is used to turn the screw head 12 of the crank 28 it will turn the Geneva wheel 30 and its pinion 32 engaging with the rack 27 of the second arm 4; this will move the second arm 4 in a linear motion along its own axis in and out of the second coupler body part 6.

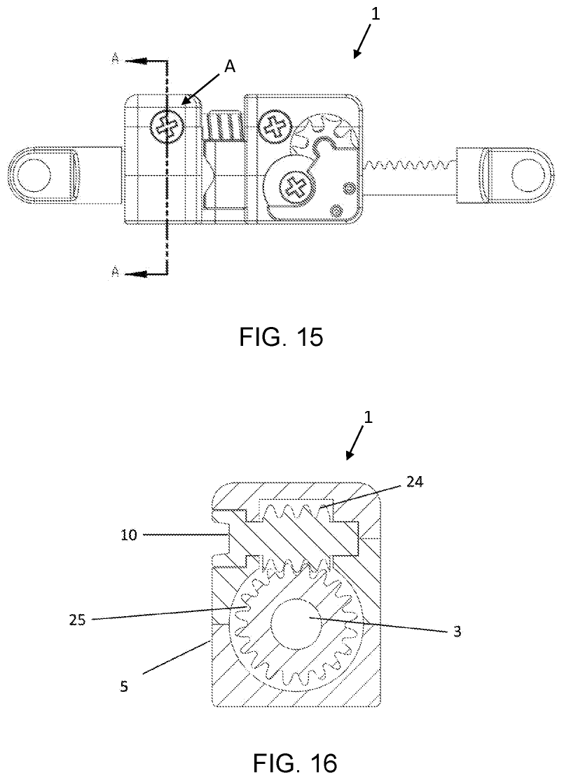

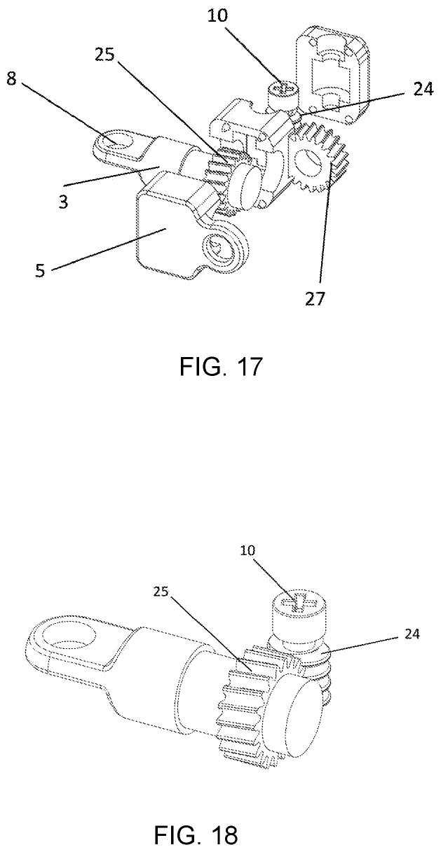

Additional details of the first joint A are provided in FIGS. 15-18. FIG. 15 is a top view of the coupler unit 1. Cross-section line A-A identifies a cross-sectional plane through the first joint or first joint mechanism A (that, in some embodiments, is well-suited for controlling axial rotation of a vertebra upon final installation). FIG. 16 illustrates the first joint A along the cross-sectional plane A-A (identified in FIG. 15) and shows the worm gear locking mechanism with the head 10 of the worm-screw 24 and the worm-wheel 25 (that is otherwise part of the first arm 3) all embedded in the first coupler body part 5. FIG. 17 is an exploded view of the coupler unit components along the section line A-A (of FIG. 15) that otherwise generate the first joint mechanism A. With additional reference to FIG. 18, once again the optional worm gear mechanism of the first joint mechanism A can include the worm 24 with the head 10, and the worm wheel 25; a manual screwdriver or a motor is applied to turn the worm 24. Turning the screw head 10 will rotate the worm 24 (driver) around its own axis which will result in turning the worm wheel 25 (driven) around the axis of first arm 3 leading to rotation its attached anchor (not shown) which is secured to the corresponding vertebra resulting in axial rotation of this vertebra as described below. With this self-locking mechanism, any force applied on the worm wheel 25 (the driven) will not result in rotating the worm 24 (the driver) which will guarantee that any correction achieved will be maintained.

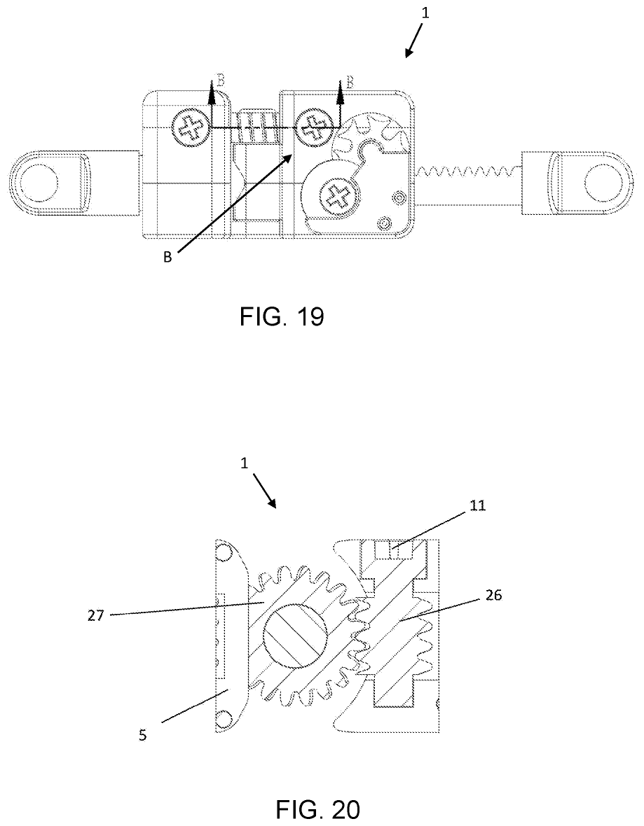

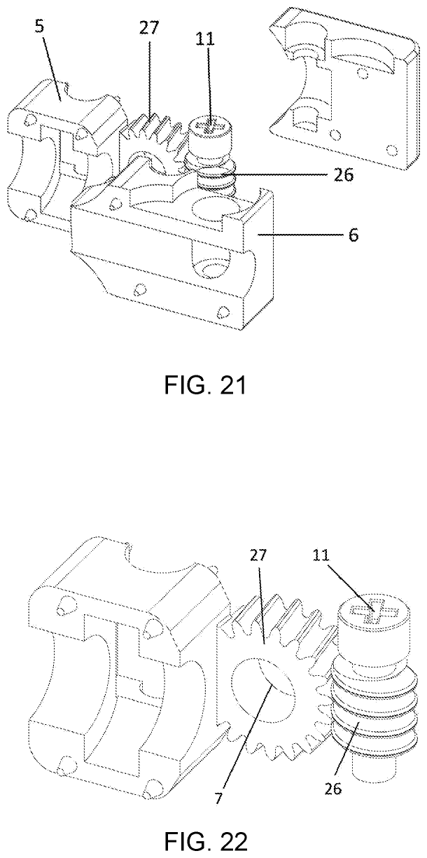

Additional details of the second joint B are provided in FIGS. 19-22. FIG. 19 is a top view of the coupler unit 1. Cross-section line B-B identifies a cross-sectional plane through the second joint or second joint mechanism B (that, in some embodiments, is well-suited for controlling sagittal angulation of a vertebra upon final installation). FIG. 20 illustrates the second joint B along the cross-sectional plane B-B (identified in FIG. 19) and shows the worm gear locking mechanism with the head 11 of the worm-screw 26 and the worm wheel 27 (that is otherwise part of the first coupler body part 5). FIG. 21 is an exploded view of the coupler unit components along the section line B-B (of FIG. 19) that otherwise generate the second joint mechanism B, as well as the outer frame of the second coupler body part 6. With additional reference to FIG. 22, once again the optional worm gear mechanism of the second joint mechanism B can include the worm 26 with the head 11, and the worm wheel 27; a manual screwdriver or a motor is applied to turn the worm 26. Turning the screw head 11 will rotate the worm 26 that in turn rotates the worm wheel 27; rotation of the worm wheel 27 rotates the first and second coupler body part 5, 6 in relation to each other around the axis 7 which will result in sagittal angulation of the first and second vertebrae attached to the first and second arms 3, 4 as described below. With this self-locking friction-based mechanism, any force applied on the worm wheel 27 (the driver) will not result in rotating the worm 26 (the driver) which will guarantee that any correction achieved will be maintained.

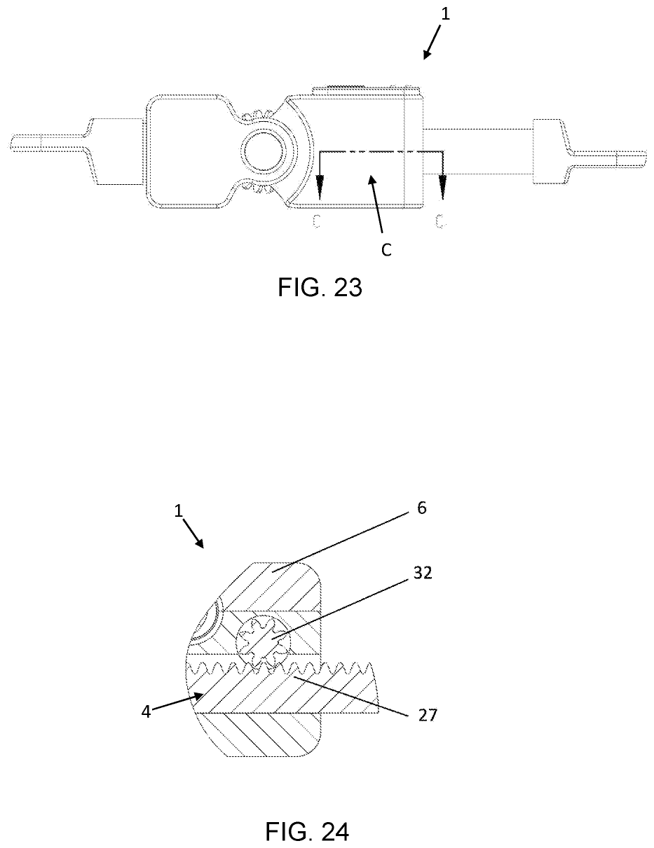

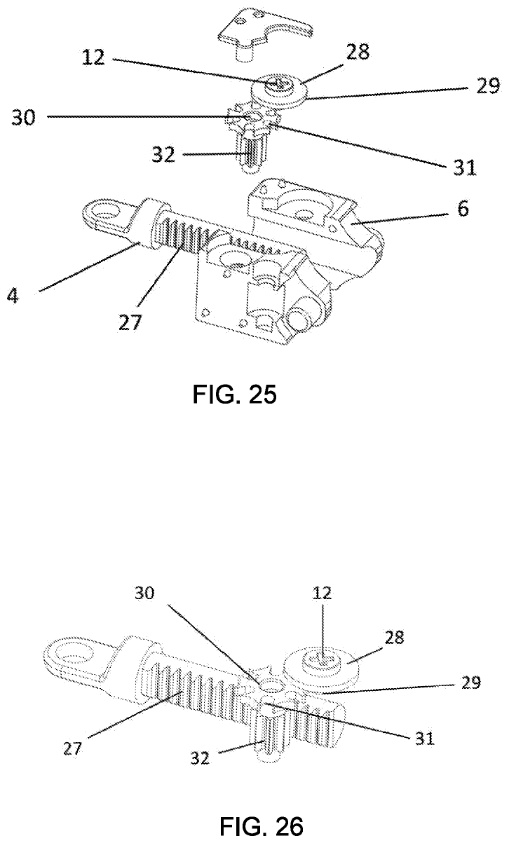

Additional details of the third joint C are provided in FIGS. 23-26. FIG. 23 is a side view of the coupler unit 1. Cross-section line C-C identifies a cross-sectional plane through the third joint or third joint mechanism C (that, in some embodiments, is well-suited for controlling coronal tilt of the vertebra upon final installation). FIG. 24 illustrates the third joint C along the cross-sectional plane C-C (identified in FIG. 23) and shows the serrated shaft 32 of the Geneva wheel engaging the ratcheted part 27 of second arm 4 both embedded in the second coupler body part 6. FIG. 25 is an exploded view of the coupler unit components along the section line C-C (of FIG. 23) that otherwise generate the third joint mechanism C, as well as the outer frame of the second coupler body part 6. With additional reference to FIG. 26, once again the cam-based locking Geneva mechanism of the joint C can include the crank 28 which carries a roller or a knob 29 and the Geneva wheel 30 with the multiple slots 31. The crank 28, which rotates by turning the screw head 12 using a manual screwdriver or a motor, carries a roller to engage with the slots 31. During one revolution of the crank 28, the Geneva wheel 30 rotates a fractional part of the revolution, the amount of which is dependent upon the number of the slots 31. The circular segment attached to the crank 28 effectively locks the wheel 30 against rotation when the roller is not in engagement and positions the wheel 30 for correct engagement of the roller with the next slot 31. The Geneva wheel 30 carries a shaft with multiple longitudinal parallel edges, these edges engage with the ratchets 27 on the second arm 4. When a manual screwdriver or a motor is used to turn the screw head 12 of the crank 28, it will turn the Geneva wheel 30 and its shaft 32 engaging with the ratcheted part 27 of the second arm 4; this will move the second arm 4 in a linear motion along its own axis in and out of the second coupler body part 6. This will result in a linear motion along the axis of the second arm 4 resulting in approximation or distraction of the first and second vertebrae attached to the first and second arms 3, 4 of the unit 1 as described below. With this self-locking mechanical based mechanism, any force applied on the Geneva wheel 30 (the driven) will not result in rotating the crank 28 (the driver) which will guarantee that any correction achieved by this mechanism will not be reversed and will be maintained.

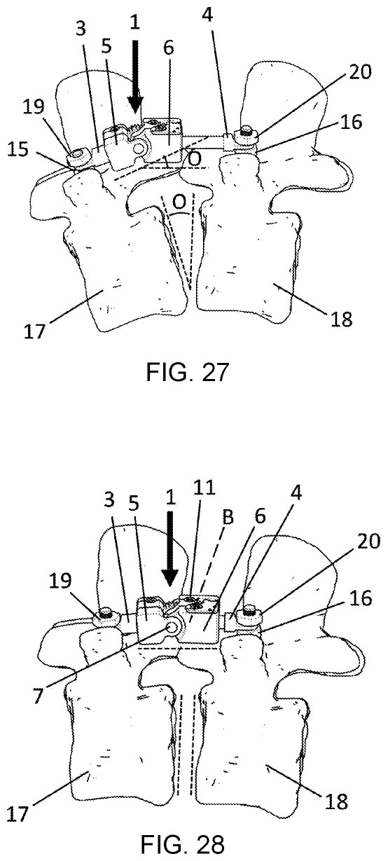

The correction-fixation coupler unit 1 can be deployed to address or correct a plethora of different spinal deformities. For example, FIG. 27 is a lateral view of a deformed vertebral motion segment with a first vertebra 17 and a second vertebra 18 sagitally angulated in relation to each other. Both vertebrae 17, 18 are connected by the correction-fixation coupler 1. The first arm 3 of the coupler 1 is attached to a first anchor 15 which is threaded and secured to the first vertebra 17. The second arm 4 of the coupler 1 is attached to a second anchor 16 which is threaded and secured to the second vertebra 18. A first nut 19 is tightened and fixes the first arm 3 to the first anchor 15; similarly, a second nut 20 is tightened and fixes the second arm 4 to the second anchor 16. In the view of FIG. 27, dashed lines parallel to a lower end plate of the first vertebra 17 and an upper endplate of vertebra 18 shows an angle of sagittal angulation O; similar dashed lines show the angle O between a lower border of the first coupler body part 5 and the second coupler body part 6.

The correction-fixation coupler unit 1 can be operated or manipulated to correct the spinal deformity of FIG. 27. For example, FIG. 28 is a lateral view of the vertebral motion segment shown in FIG. 27 after correction of the sagittal angulation using the correction-fixation coupler 1 (that again connects the first vertebra 17 to the second vertebra 18). The first and second dashed vertical lines in FIG. 28 correspond to the lower endplate of the first vertebra 17 and the upper endplate of the vertebra 18 and are now parallel to each other after correction of the sagittal angulation of the vertebrae 17, 18. The horizontal dashed line in FIG. 28 is parallel to the lower end of the coupler unit 1 and reflects that the lower border of the first coupler body part 5 and the lower border of the second coupler body part 6 are now aligned leading to alignment of the first vertebra 17 and the second vertebra 18. This motion was done by turning the second actuator/screw head 11 to mobilize the second joint B embedded partly in second coupler body part 6 around the axis 7, thereby aligning the first and second coupler body parts 5, 6 in relation to each other.

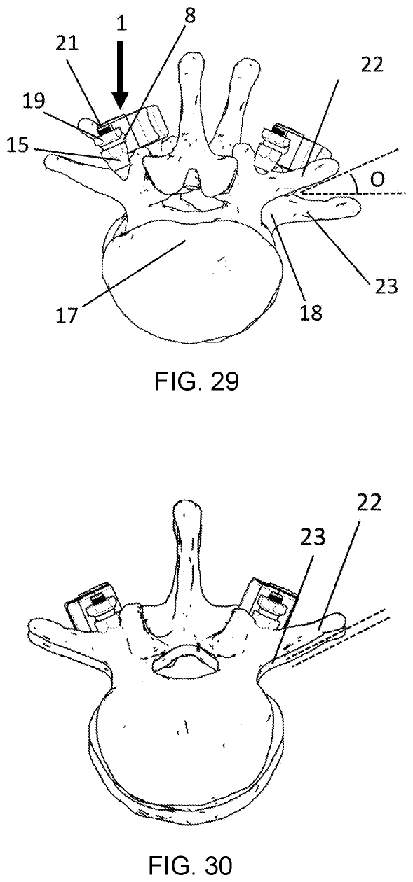

Another example of spinal deformity corrections available with the systems and methods of the present disclosure is reflected by FIG. 29 that provides a top view of a deformed vertebral motion segment with the first vertebra 17 and the second vertebra 18 axially rotated in relation to each other. The correction-fixation coupler 1 has been initially connected to the vertebrae 17, 18. A post 21 of an anchor (e.g., pedicle screw) 15 has been passed through the aperture 8 (referenced generally) of the first arm 3 (best shown in FIG. 9) of the coupler unit 1, and a nut 19 tightened to fix the first arm 3 to the anchor 15. Dotted lines parallel to a transverse process 22 of the first vertebra 17 and a transverse process 23 of the second vertebra 18 shows the angle of axial rotation O.

The correction-fixation coupler unit 1 can be operated or manipulated to correct the spinal deformity of FIG. 29. For example, FIG. 30 is a top view of vertebral motion segment shown in FIG. 29 after correction of the axial rotation using the correction-fixation coupler 1 (that again connects the first vertebra 17 to the second vertebra 18). The dotted lines corresponding to the transverse process 22 of the first vertebra 17 and the transverse process 23 of the second vertebra 18 are now parallel to each other after correction of the axial rotation by the correction fixation coupler unit 1, resulting in alignment of the vertebrae 17, 18.

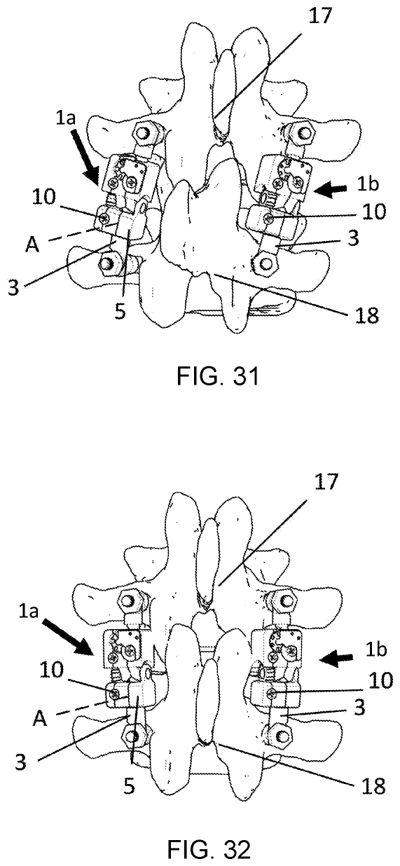

In some examples, two of the correction-fixation coupler units of the present disclosure can be installed or deployed to correct spinal deformities. For example, FIG. 31 is a posterior view of the deformed the vertebral motion segment shown in FIG. 29 with the first vertebra 17 and the second vertebra 18 axially rotated in relation to each other. In the initial state of FIG. 31, both vertebrae 17, 18 are connected by opposing correction-fixation coupler units (labeled 1a and 1b). One or both of the units 1a, 1b can be operated or manipulated to correct the spinal deformity of FIG. 31. For example, FIG. 32 is a posterior view of the vertebral motion segment shown in FIG. 31 showing the correction of the axial rotation using one or both of the correction-fixation coupler units 1a, 1b (otherwise connecting the first vertebra 17 to the second vertebra 18). This correction was done by turning the first actuator/screw head 10 to mobilize the first joint A embedded in first coupler body part 5 of the corresponding unit 1a, 1b, rotating the corresponding first arm 3 around its own axis.

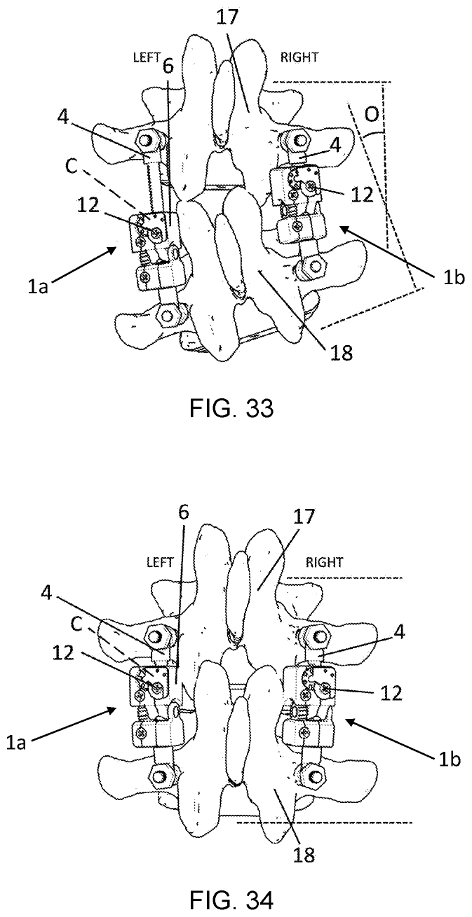

Another example of spinal deformity corrections available with the systems and methods of the present disclosure is reflected by FIG. 33 that provides a posterior view of a deformed vertebral motion segment with the first vertebra 17 and the second vertebra 18 coronally tilted in relation to each other. In the initial state of FIG. 33, both vertebrae 17, 18 are connected by first and second correction-fixation coupler units 1a, 1b. In the view, dotted lines parallel to an upper end plate of the first vertebra 17 and parallel to a lower endplate of second vertebra 18 shows the angle of coronal tilt O.

One or both of the correction-fixation coupler units 1a, 1b can be operated or manipulated to correct the spinal deformity of FIG. 33. For example, FIG. 34 is a posterior view of the vertebral motion segment shown in FIG. 33 after correction of the coronal tilt using correction-fixation coupler units 1a, 1b otherwise connecting the first vertebra 17 to the second vertebra 18. This correction was done by turning third actuator/screw head 12 on the first or left side unit la to mobilize the corresponding third joint C embedded in the second coupler body part 6 to move the corresponding second arm 4 linearly inside the second coupler body part 6 to shorten it, while turning third actuator/screw head 12 on the second or right side unit 1b to mobilize the corresponding third joint C to move the corresponding second arm 4 linearly outside second coupler body part 6 to elongate the second arm 4 until the two vertebrae 17, 18 align with each other. As reflected by dotted lines corresponding to the upper end plate of the first vertebra 17 and the lower endplate of the second vertebra 18 are now parallel to each other after correction of the coronal tilt and alignment of the vertebrae 17, 18. Regarding coronal tilt, despite it being straightforward for one of ordinary skill in the art to add a joint mechanism in the correction fixation coupler to control and correct the coronal tilt of the deviated vertebra, the inventors of the present disclosure have found that the axis or rotation of such a joint mechanism will be rotation around the Z/Anteroposterior axis which is the same axis of rotation of the pedicle screw at which the coupler unit is attached. Operating such a joint can turn the pedicle screw around its own axis within the pedicle which may lead to unwanted and potentially risky tightening or loosening of the screw. Therefore, in some embodiments, coronal tilt can be corrected using the third joint mechanism C of the coupler units 1a, 1b bilaterally.

In some embodiments, the coupler unit 1 is well-suited for scoliosis correction procedures. Scoliosis, the most common spinal deformity, is a three-dimensional (3D) deformity that can be associated with lateral deviation in the coronal plane, thoracic hypokyphosis in the sagittal plane and rotation in the axial plane. A correction fixation coupler intended to correct such a deformity should include the joint mechanisms capable of correcting axial rotation and/or, sagittal angulation and/or coronal tilt. In some embodiments, the coupler unit 1 can be utilized for scoliosis correction and thus can optionally referred to as “the scoliosis correction-fixation coupler”; however, from the practical application there will be a whole system with many coupler configurations covering all the possible combination of joint mechanisms capable of correcting any type of vertebral deviation according to the need of each specific case and each specific intervertebral segmental deformity.

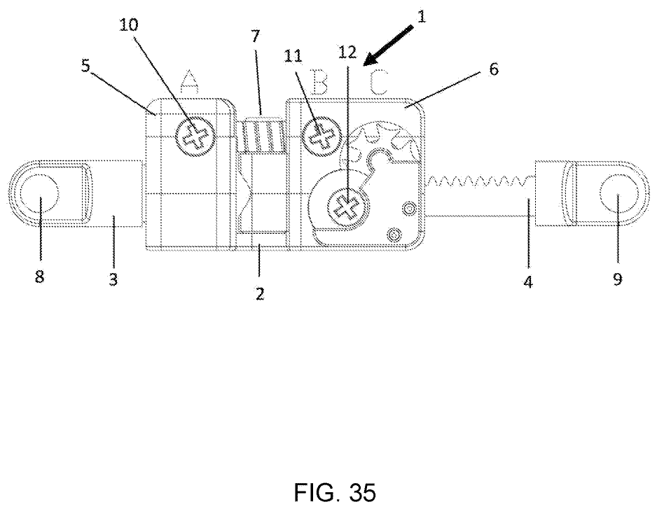

With the above in mind, and commensurate with the descriptions above, FIG. 35 is a top view identifying examples of one configurations of the scoliosis correction-fixation coupler unit including the three joint mechanisms A, B, C. The first joint A is controlled by the actuator/screw head 10 to mobilize the second arm 4 to rotate around itself resulting in correction of axial rotation of the first vertebra in relation to the second vertebra. The second joint B is controlled by actuator/screw head 11 mobilize first coupler body part 5 in relation to the second coupler body part 6 (and vice-versa) around the axis 7 resulting in correction of sagittal angulation of the first vertebra in relation to the second vertebra. The third joint C is controlled by the actuator/screw head 12 to mobilize the second arm 4 linearly in and out of the second coupler body part 6 along its own axis. This motion can compress and distract the first and second vertebrae in relation to each other correcting the cranio-caudal linear displacement. When two of the coupler units 1 are attached bilaterally to the right and left sides of the vertebral motion segment; when the second arm 4 attached to the pedicles on the right side posteriorly is distracted and the second arm 4 attached to pedicles on the left side is compressed or vice versa, the motion of the third joint C on both sides sequentially will result in correction of coronal tilt of the first vertebra and second vertebrae in relation to each other both clockwise and anticlockwise. The coupler unit 1 in FIG. 35 allows the correction of deviation of the two vertebrae in four of the six axes of motion: axial rotation, sagittal angulation, coronal tilt, and linear compression and distraction. This can correct the main vertebral deviation occurring either in scoliosis or kyphosis. The two remaining motion deviations (namely anteroposterior translation and mediolateral translation) are not commonly present in scoliotic deformity each will have its own separate joint mechanisms D and E capable of correcting each of these deviations respectively and they will be shown later.

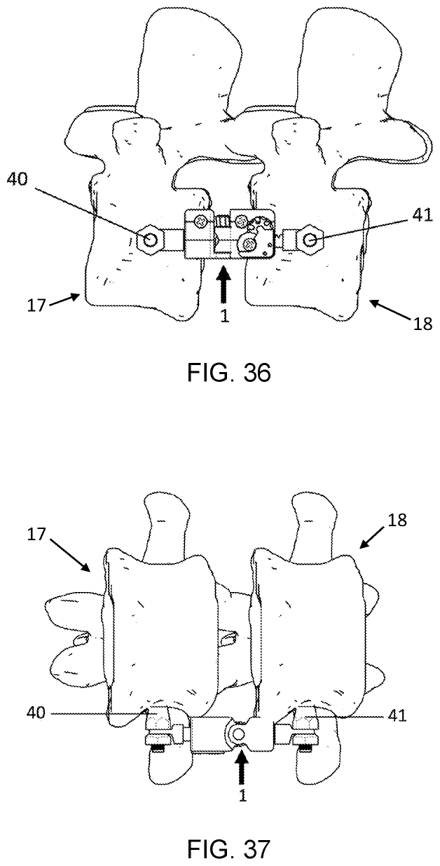

While several of the embodiments described above implicate posterior transpedicular insertion of the coupler unit 1, other approach techniques and installations are also acceptable, such as anterior insertion (e.g., from the front and anchored to the side of the vertebral bodies). For example, FIG. 36 is lateral view of a vertebral motion segment showing the correction-fixation coupler unit 1 connecting the first and a second vertebra 17, 18. The coupler unit 1 is attached proximally and distally to a first vertebral body screw 40 and a second vertebral body screw 41, both inserted in the lateral side of the corresponding vertebral body. In another example, FIG. 37 is an anterior view of a vertebral motion segment showing the correction-fixation coupler unit 1 connecting the first and a second vertebra 17, 18. The coupler unit 1 is attached proximally and distally to a first vertebral body screw 40 and second vertebral body screw 41, both inserted in the lateral side of the corresponding vertebral body.

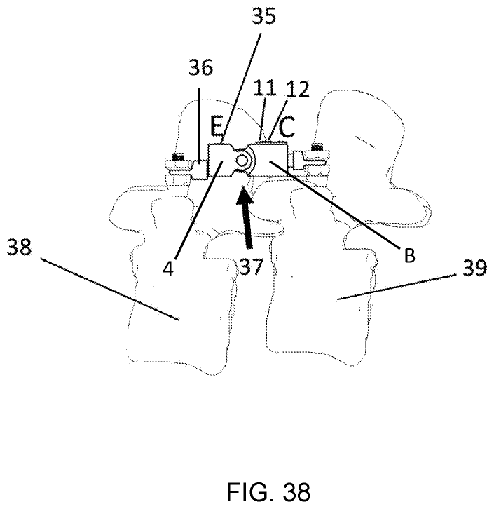

As mentioned above, the coupler units of the present disclosure can incorporate one or more of five different types of joints or joint mechanism. The coupler unit 1 described above provides three joints (designated as joint type A, joint type B, and joint type C). Other spinal deformities may implicate other formats of coupler unit joint configurations or selections differing from those of the coupler unit 1 (that can otherwise be appropriate for addressing or correction typical scoliosis-related deformities). For example, another coupler unit 37 in accordance with principles of the present disclosure is shown in FIG. 38 as installed to a vertebral motion segment. The coupler unit 37 can be highly akin to the coupler unit 1 (FIG. 9) described above, and includes opposing arms (one of which is labeled at 36) movably maintained by a coupler body part (e.g., the first coupler body part 5) and three of the uniaxial joints of the present disclosure, in particular joint types B, C, and E. In some embodiments, the coupler unit 37 can be well-suited for correcting spondylolisthesis deformities. As a point of reference, lumbar spondylolisthesis or anterolithesis is a clinical condition where the main vertebral deviations are anterior translation of the proximal vertebra in relation to the distal vertebra, narrowing of the disc space, and loss of lumbar lordosis. A coupler correcting these three deviations can be named “spondylolisthesis coupler” as it will include three joint mechanisms capable of correcting these three deviations. For example, in the view of FIG. 38, a first vertebra 38 anteriorly translated on a second vertebra 39. The coupler unit 37 is attached to the first vertebra 38 and the second vertebra 39. The configuration of correction-fixation coupler unit 37 includes the joint mechanisms chosen to correct the main vertebral deviations in spondylolisthesis deformity namely anterior translation, narrowing of the disc space, and loss of lumbar lordosis. Turning actuator (e.g., screw head) 35 (referenced generally) controls the joint E to move the arm 36 in a linear motion along the sagittal plane/Z axis in a posteriorly/backward direction correcting anterior translation of the first vertebra 38 aligning it with the second vertebra 39. Turning actuator (e.g., screw head) 11 controls the joint mechanism B to correct sagittal angulation to restore lumbar lordosis. Turning actuator (e.g., screw head) 12 controls the joint mechanism C to linearly move and elongate the arm 36 by moving it out of coupler body part 6 which if operated bilaterally will distract both vertebrae 38, 39 in relation to each other correcting the narrowing of disc space.

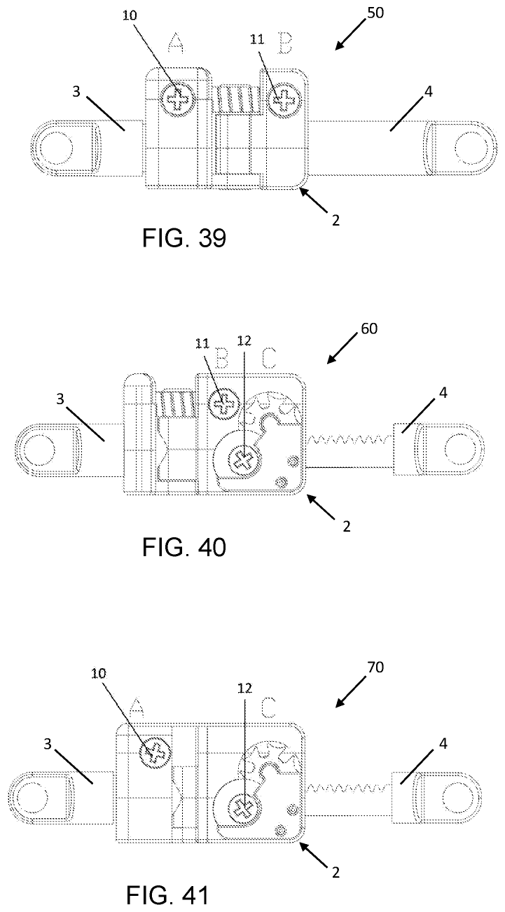

The coupler units 1, 37 described above are but two non-limiting examples in accordance with principles of the present disclosure, both incorporating three different joint types (e.g., joint types A, B, C with coupler unit 1; joint types B, C, E with coupler unit 37). Other three joint type coupler units are also acceptable (i.e., incorporating a different combination of the joint types A, B, C, D, and E). In yet other embodiments, coupler units of the present disclosure can incorporate or include four or five of the joint types A, B, C, D, and E. In other embodiments, coupler units of the present disclosure can include or incorporate two of the joint types A, B, C, D, or E. By way of non-limiting examples, FIG. 39 illustrates another coupler unit 50 in accordance with principles of the present disclosure. The coupler unit 50 can be highly akin to the coupler units described above, and generally includes the coupler 2 maintaining the first and second arms 3, 4. In addition, the coupler unit 50 provides two joints or joint mechanisms, and in particular the joint type A and the joint type B. Commensurate with the descriptions above, upon final deployment to a vertebral motion segment, turning the actuator/screw head 10 associated with the joint A operates to correct axial rotation. Turning the actuator/screw head 11 associated with the joint B operates to correct sagittal angulation.

FIG. 40 illustrates another coupler unit 60 in accordance with principles of the present disclosure incorporating a two joint configuration. The coupler unit 60 can be highly akin to the coupler units described above, and generally includes the coupler 2 maintaining the first and second arms 3, 4. In addition, the coupler unit 60 provides two joints or joint mechanisms, and in particular the joint type B and the joint type C. Commensurate with the descriptions above, upon final deployment to a vertebral motion segment, turning the actuator/screw head 11 associated with the joint B operates to correct sagittal angulation. Turning the actuator/screw head 12 associated with the joint C operates to correct compression and distraction as well as coronal tilt.

FIG. 41 illustrates another coupler unit 70 in accordance with principles of the present disclosure incorporating a two joint configuration. The coupler unit 70 can be highly akin to the coupler units described above, and generally includes the coupler 2 maintaining the first and second arms 3, 4. In addition, the coupler unit 70 provides two joints or joint mechanisms, and in particular the joint type A and the joint type C. Commensurate with the descriptions above, upon final deployment to a vertebral motion segment, turning the actuator/screw head 10 associated with the joint A operates to correct axial rotation. Turning the actuator/screw head 12 associated with the joint C operates to correct compression and distraction as well as coronal tilt

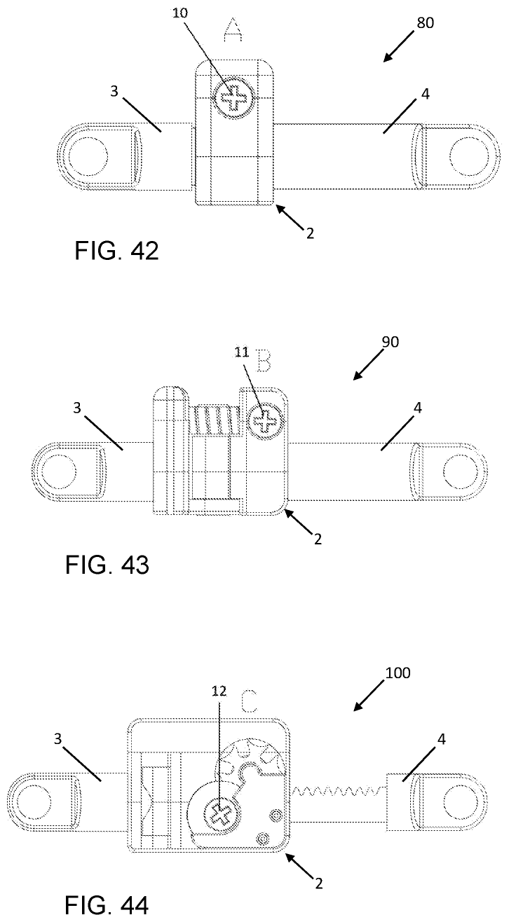

In yet other embodiments, coupler units of the present disclosure can include or incorporate a single one of the joint types A, B, C, D, or E. By way of non-limiting example, FIG. 42 illustrates another coupler unit 80 in accordance with principles of the present disclosure incorporating a single joint configuration. The coupler unit 80 can be akin to the coupler units described above, and generally includes the coupler 2 maintaining the first and second arms 3, 4. In addition, the coupler unit 80 provides a single joint or joint mechanism, and in particular the joint type A. Commensurate with the descriptions above, upon final deployment to a vertebral motion segment, turning the actuator/screw head 10 associated with the joint A operates to correct axial rotation.

FIG. 43 illustrates another coupler unit 90 in accordance with principles of the present disclosure incorporating a single joint configuration. The coupler unit 90 can be akin to the coupler units described above, and generally includes the coupler 2 maintaining the first and second arms 3, 4. In addition, the coupler unit 90 provides a single joint or joint mechanism, and in particular the joint type B. Commensurate with the descriptions above, upon final deployment to a vertebral motion segment, turning the actuator/screw head 11 associated with the joint B operates to correct sagittal angulation.

FIG. 44 illustrates another coupler unit 100 in accordance with principles of the present disclosure incorporating a single joint configuration. The coupler unit 100 can be akin to the coupler units described above, and generally includes the coupler 2 maintaining the first and second arms 3, 4. In addition, the coupler unit 100 provides a single joint or joint mechanism, and in particular the joint type C. Commensurate with the descriptions above, upon final deployment to a vertebral motion segment, turning the actuator/screw head 12 associated with the joint C operates to correct compression and distraction as well as coronal tilt.

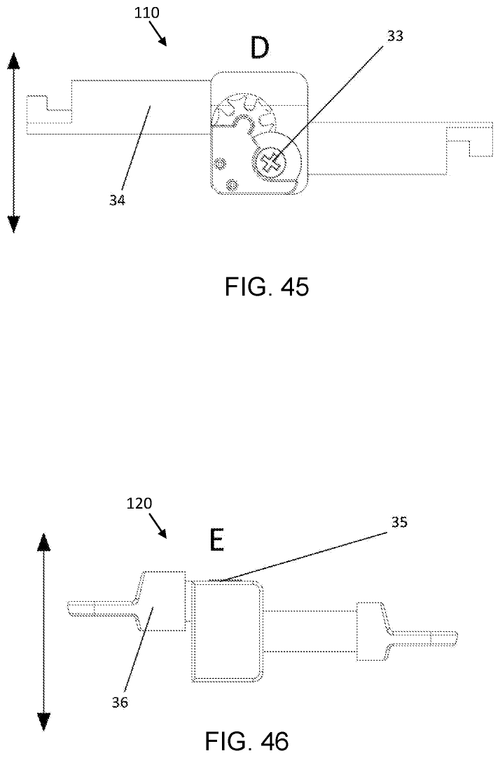

FIG. 45 illustrates another coupler unit 110 in accordance with principles of the present disclosure incorporating a single joint configuration. The coupler unit 110 can be akin to the coupler units described above, and generally includes the coupler maintaining the first and second arms (one of which is labeled at 34). In addition, the coupler unit 110 provides a single joint or joint mechanism, and in particular the joint type D including an actuator (e.g., screw head 33). Commensurate with the descriptions above, the joint type D can provide control over medio-lateral translation upon final deployment to a vertebral motion segment. Turning the actuator/screw head 33 associated with the joint D will move the arm 34 in a linear motion along the coronal plane/X axis which will mobilize the arm 34 to the right or the left correcting medio-lateral translation of the vertebra.

FIG. 46 illustrates another coupler unit 120 in accordance with principles of the present disclosure incorporating a single joint configuration. The coupler unit 120 can be akin to the coupler units described above, and generally includes the coupler maintaining the first and second arms (one of which is labeled at 36). In addition, the coupler unit 120 provides a single joint or joint mechanism, and in particular the joint type E including an actuator (e.g., screw head 35). Commensurate with the descriptions above, the joint type E can provide control over antero-posterior translation upon final deployment to a vertebral motion segment. Turning the actuator/screw head 35 associated with the joint E will move the arm 36 in a linear motion along the sagittal plane/Z axis which will mobilize the arm 36 anteriorly or posteriorly correcting antero-posterior translation of the vertebra.

In some embodiments and with any of the coupler units of the present disclosure, to achieve individual intervertebral segmental correction of vertebral deviation, two of the coupler units are attached to the two adjacent vertebrae and the correction motion will occur at a single intervertebral disc level. In some cases, including congenital fusion of two vertebrae or a very narrow space between the two adjacent concave pedicle screws around the apex of scoliosis in dorsal spine, one or more levels can be skipped according to the need. With these and related embodiments, the correction motion will occur collectively at two or three or more intervertebral discs levels with the aim of aligning the most proximal and the most distal vertebrae where the coupler unit is attached. The site of insertion of the coupler units can be determined case by case depending on many factors including the age of the patient, size of the vertebrae, presence of congenital vertebral anomalies, type of deformity, severity of the curve, site of the deformity among many other factors. Therefore, there is no predetermined “recipe” for the coupler unit arrangement in different clinical scenarios. The following descriptions are only a few non-limiting examples of possible coupler unit arrangements in long curve deformities.

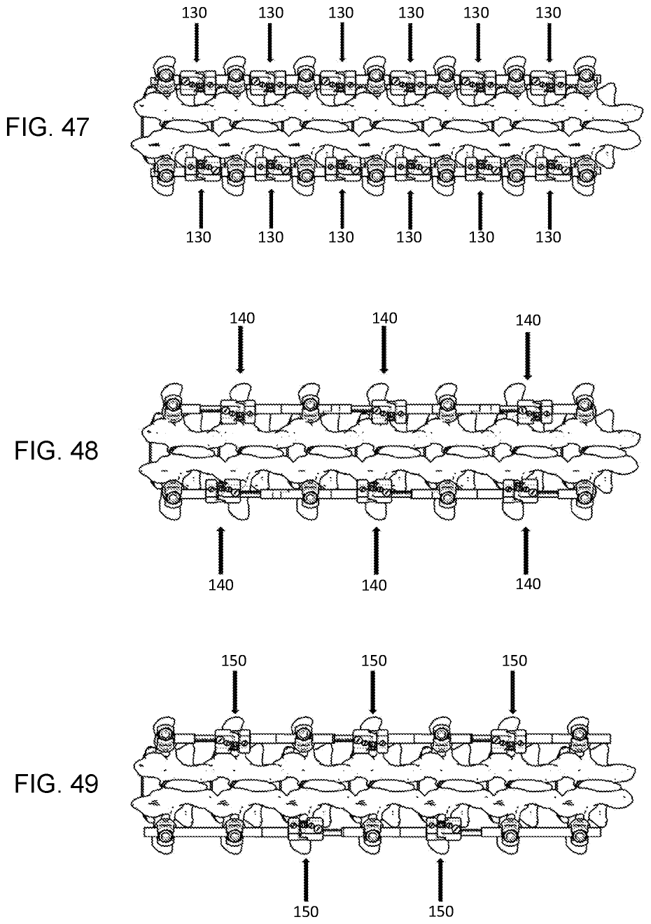

FIG. 47 is a posterior view of a vertebral column with an example of arrangement of multiple coupler units 130 inserted posteriorly and attached to pedicle screws using a top-loading system. The coupler units 130 can assume any of the forms of the present disclosure (e.g., the coupler unit 1) and in FIG. 47, have been inserted at each single intervertebral levels bilaterally without skipping any vertebra.

FIG. 48 is a posterior view of a vertebral column with another example of arrangement multiple coupler units 140 inserted posteriorly and attached to pedicle screws using a top-loading system. The coupler units 140 can assume any of the forms of the present disclosure (e.g., the coupler unit 1) and in FIG. 48, the coupler units 140 have been inserted at every other level in a symmetrically bilateral (mirror image) arrangement. Each coupler unit 140 will correct the deformity occurring collectively at two adjacent intervertebral discs with a non-instrumented vertebra in-between.

FIG. 49 is a posterior view of a vertebral column with another example of arrangement multiple coupler units 150 inserted posteriorly and attached to pedicle screws using a top-loading system. The coupler units 150 can assume any of the forms of the present disclosure (e.g., the coupler unit 1) and in FIG. 49, the coupler units 150 have been inserted at every other level in non-symmetrical bilateral (non-mirror image) alternating arrangement. In this arrangement, each vertebral body is only instrumented and attached to an arm of a coupler unit 150 on either the right or left pedicle and the contralateral pedicle is non-instrumented.

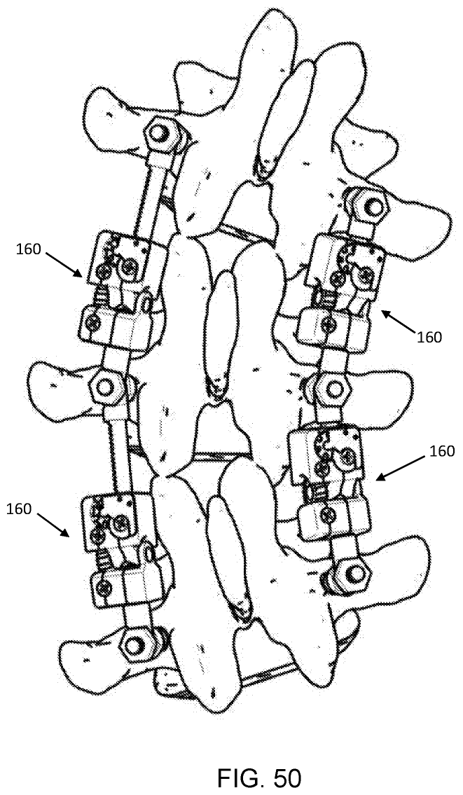

FIG. 50 is a posterior view of a spinal deformity showing coronal tilt at two levels (three vertebrae and two vertebral motion segments). Four correction fixation coupler units 160 are attached to their corresponding pedicle screws, two on each side of the spine. The coupler units 160 can assume any of the forms of the present disclosure, for example akin to the coupler unit 1 (FIG. 9). In the arrangement of FIG. 50, the coupler units 160 are ready to be manipulated or operated to correct the individual vertebral deviations at the intervertebral junctions.

In some embodiments, the systems of the present disclosure, including the correction-fixation coupler units, can in some clinical settings be used as a temporary appliance to correct the spinal deformity and then be removed after the corrected spine being fixed by another fixation apparatus or a contoured conventional solid rod arrangement. The use of the some embodiments of the present disclosure as an appliance used to temporarily correct the spinal deformity and removed from the patient after correction may allow the repetitive use of the correction-fixation coupler unit(s) similar to any spinal instrument that could be sterilized and used in many patients, which can reduce the costs significantly among many other possible benefits from having this additional option. To use the systems or units of the present disclosure temporarily, it can be be combined with another fixation apparatus designed to be able to connect the spine easily in its deformed position and once the spine is corrected the apparatus is locked to fix the spine in its corrected position allowing the removal of correction fixation coupler unit(s). The systems or units of the present disclosure can also be used temporarily in combination with conventional solid rods (not shown in the Figures); once the spine is corrected to the required position by the correction-fixation coupler unit(s) as described above, a contoured solid rod that conforms with the spine's new corrected position can be connected and attached to the previously-placed screws (otherwise utilized to mount the coupler unit(s) to the various ones of the vertebrae) to lock the spine in its corrected position allowing the removal of correction fixation coupler unit(s).

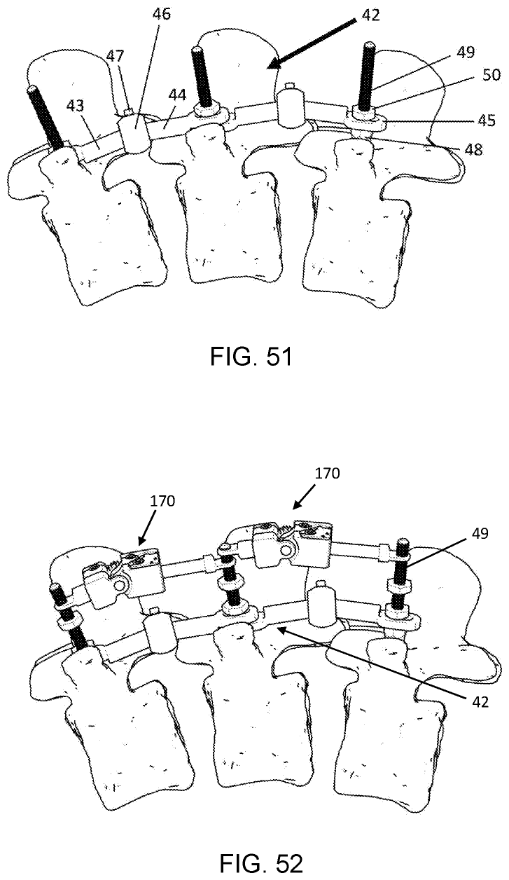

One non-limiting example of use of the coupler units of the present disclosure as a temporary appliance, begins with FIG. 51. FIG. 51 provides a lateral view of a deformed spine segment including the vertebrae with a kyphotic deformity. At a first step represented by FIG. 51, a fixation apparatus 42 has been connected to posts 49 of side loading pedicle screws 48 secured in the vertebrae. The fixation apparatus 42 comprises multiple poly-axial/multiaxial connectors; each connector has a first arm 43 and a second arm 44 to connect to the pedicle screws 48 through an oval or elliptical aperture 45 (referenced generally) which allow sliding of the post 49 of the screw 48 in a cranio-caudal direction to accommodate any compression distraction between the vertebrae. Each of the connectors further includes a lockable poly-axial/multiaxial articulated joint mechanism 46 which when unlocked/loose will allow arms 43 and 44 to connect with two deviated vertebrae within a deformed intervertebral segment easily without any stress. The poly-axial/multiaxial articulated joint mechanism 46 could be a ball and socket or any similar joint allowing such a multiplanar motion; this articulated joint 46 comprises a locking means 47 for selectively locking the articulated joint. The locking means 47 is arranged to provide a plurality of locking conditions to the articulated joint 46, with the locking conditions including a fully unlocked condition, a partially locked condition, and a fully locked condition. The articulated joint 46 will be fully unlocked (loose) while connecting the apparatus connector to the pedicle screws 48 secured in the deviated vertebrae, to allow for its easy attachment. Once the spinal deformity is corrected by the correction-fixation coupler units as described below, the locking means 47 will be used to fully lock in the corrected position to fix the aligned spine. In the state of FIG. 51, the articulated joints 46 are fully unlocked (loose) and a nut 50 of the post 49 of the pedicle screw 48 is not fully tightened to allow for the post to move craniocaudally within the elliptical aperture 45 of the arms 43, 44 of the connector to accommodate any subsequent linear corrective motion in this direction.

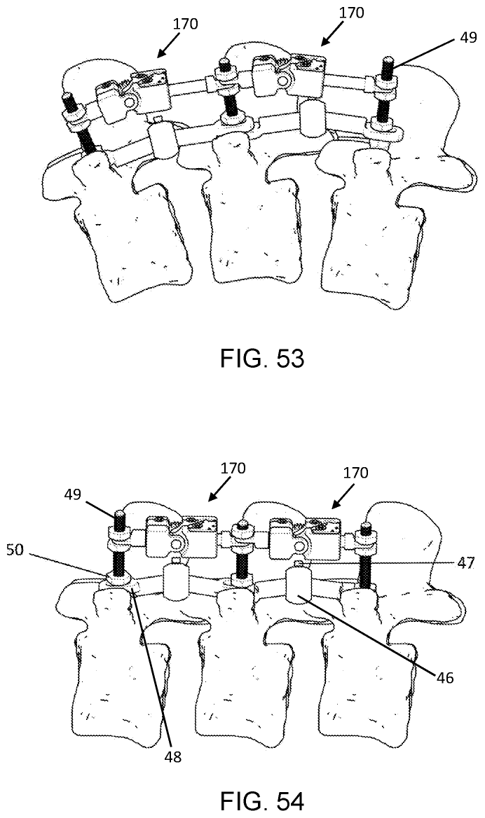

FIG. 52 illustrates completion of a subsequent (e.g., second) step. In particular, FIG. 52 is a lateral view of a deformed spine segment and depicts a loose fixation apparatus connected to the posts of a side loading pedicle screws secured in the vertebrae. Correction-fixation coupler units 170 in accordance with principles of the present disclosure have been connected to the pedicle screw posts 49 on top of the loose fixation apparatus 42. In the view of FIG. 53, the coupler units 170 have been secured and fixed to the pedicle screw posts 49 upon completion of a subsequent (e.g., third) step.

FIG. 54 illustrates completion of a subsequent (e.g., fourth) step. In particular, FIG. 54 is a lateral view of the spine segment after correction using the correction-fixation couplers units 170. Both the locking means 47 of the articulated joint 46 of the fixation apparatus 42 and the nuts 50 securing it to the post 49 of pedicle screws 48 are still loose.

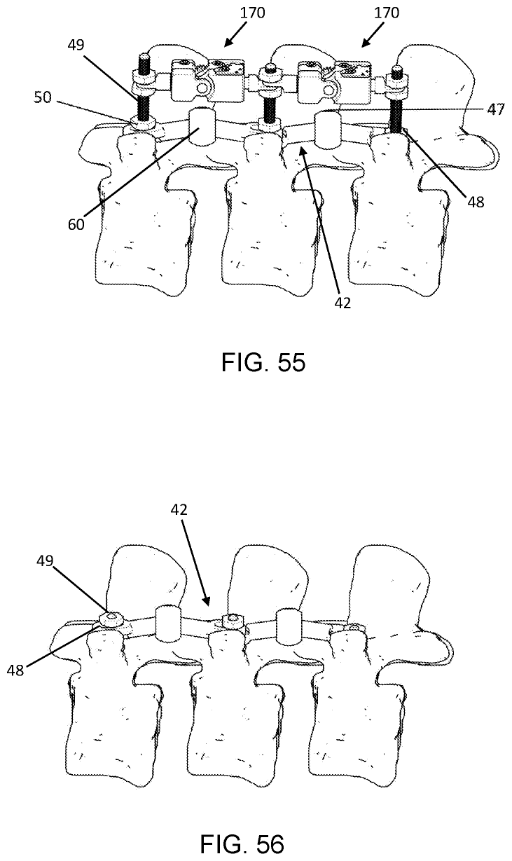

FIG. 55 illustrates completion of a subsequent (e.g., fifth) step. In particular, FIG. 55 is a lateral view of the spine segment after correction using the correction-fixation coupler units 170. Both the locking means 47 of the articulated joints 60 of the fixation apparatus 42 and the nuts 50 securing it to the post 49 of pedicle screws 48 are now tightened fully to fix the articulated joint 60 in the corrected position and fix the fixation apparatus 42 in the posts 49 of pedicle screws 48. Now the fixation apparatus 42 is fully locked keeping the corrected spine fixed in its aligned position. Finally, FIG. 56 illustrations completion of a subsequent (e.g., sixth) step, and is a lateral view of spine segment after correction now fixed in its aligned position by the fixation apparatus 42 after removal of the correction fixation coupler units 170 (FIG. 55) and cutting the protruding parts of the posts 49 of the pedicle screws 48.

The systems, coupler units, and methods of the present disclosure provide a marked improvement over previous designs. In some embodiments, the screws anchored to the deviated vertebrae in a spinal deformity are connected via multidirectional couplers that can be configured to any position to fit the current deformity allowing easy and smooth connection between the deviated screws. This decreases significantly any stress on the screws and hence the screw bone interface. In some embodiments, the systems and coupler units of the present disclosure avoids the need to maneuver and rotate a stiff rod and nullify the need for in-situ bending after attaching it the spine; this will avoid the force applied on different areas of the bone implant interface preventing the risks and failures associated with such maneuvers. Some systems of the present disclosure are designed to measure the amount of force/torque needed to correct each vertebra in any specific direction preventing any unwanted non-calculated stress on the spine implant interface. With the current advancement in radiology and 3D measurement and printing, vertebral deviations can be transformed into accurate numbers; these numbers can be executed accurately using the current device and methods. This process can be called “digitization of spinal deformity”. By allowing each level and each direction to be corrected independently, the some systems and methods of the present disclosure allow the gradual correction of different levels at different directions depending on the angles and distances measured and on the amount of torque needed. This allows starting with a certain torque and use the driver in different levels and different directions within this predetermined torque and then increase the torque gradually to do a simultaneous sequential correction at many levels and directions going back and forth between them. This will allow the minimum force applied at different levels for correction and taking advantage of tissue yielding occurring at different levels.

Spinal deformity has always been dealt with as a global deformity in multiple vertebrae however from the mechanical accurate analysis it should be dealt with as multiple separate (but interconnected) deformities, each occurring at a single vertebral segment (two adjacent vertebrae with an intervening disc). With this in mind, systems of the present disclosure are the first that can allow true segmental 3D correction of each individual vertebra within the curve. This correction is done not only in each vertebra independent to the other vertebrae of the curve; but it is done for each direction of deviation either it is rotation or linear independent from the other deviations in the same vertebra. Previous studies have shown that within the same curve some levels have deformities in some axes significantly more than the others, and some levels have deformities in only one single axis. The system allows correction of a single vertebra along a single intended specific axis of motion independent from the other axes within the same segment. Systems of the present disclosure allow correction of the deformity at each vertebral segment in different axes of motions independent from the other segments within the same curve. The disclosed methods allow the exact measurement of each vertebral deviation in the six axes of motion of the coordinate system. Having the local coordinates of each vertebra allow the exact measurement of its deviation. For an intervertebral segment by measuring the deviation of the two vertebrae will allow the amount required for correction in each of the six directions of deviation.

The systems of the present disclosure, including the coupler, arms and the vertebral anchors, can be readily fabricated the same way as many of the orthopedic and spinal implants are already fabricated and used. The joint mechanisms are based on the so-called robotic uniaxial joints that are already in use in many robotic and industrial applications in different fields. The self-locking mechanism is based on mechanical locking, friction locking or singularity locking; all are known mechanisms in the industry. The material used to fabricate the system will be metal including stainless steel or most probably titanium or cobalt to be compatible with the MRI. All these materials has been used in orthopedic and spinal implants for decades now without significant risk.

Although the present disclosure has been described with reference to preferred embodiments, workers skilled in the art will recognize that changes can be made in form and detail without departing from the spirit and scope of the present disclosure.

1-30. (canceled)

31. A vertebral system for correction and fixation of spinal deformities comprising:
at least one correction-fixation coupler unit including:
a coupler body with a first end and a second end;
wherein the coupler body includes at least one body part;
a first arm extending from the first end;
a second arm extending from the second end;
wherein the first arm is configured for attachment to a first vertebra via a first anchor, and the second arm is configured for attachment to a second vertebra via a second anchor; and
one or more articulating joint mechanisms carried by the coupler body;
wherein each joint mechanism is configured to enable the first arm and the second arm to move relative to one another such that following installation to the first and second vertebra, the joint mechanism is operable to manipulate the first and second vertebrae relative to one another in at least one of 6 degrees of freedom to realign the first vertebra and the second vertebra resulting in correction of the vertebral deviation, and further wherein the at least one coupler unit collectively with all its possible different joint mechanisms configurations can allow the motion in any of twelve directions of motion including six rotations (three clockwise and three anticlockwise) and six linear displacements (three positive and three negative).

32. The vertebral system of claim 31, wherein the at least one joint mechanism comprises a scale indicating at least one of an amount of rotation and a linear displacement occurring at the joint mechanisms and the arms.

33. The vertebral system of claim 31, further comprising at least one of a manual driver and a motor configured to apply a driving force onto the at least one joint mechanism, and further wherein the manual driver or motor includes a means to measure a torque or force applied to mobilize the at least one joint mechanism.

34. The vertebral system of claim 31, wherein the at least one coupler unit further includes at least one of posts and markers along at least one of the arms and the body parts to measure the vertebral deviation before, during and after correction intraoperatively.

35. A method of treating a spinal deformity, comprising correcting individual vertebral deviations with the steps of:
using a motion control coordinate system to calculate individual vertebral deviations within the spinal deformity in all six degrees of freedom: three linear along X, Y, and Z axes, and three rotational around the X, Y, and Z axes;
determining deviation parameters between first and second vertebrae of the spinal deformity in terms of an axis of rotation or translation, direction of rotation clockwise or anticlockwise, direction of translation positive or negative, and an amount of rotation in degrees and of translation in millimeters;
selecting a first correction-fixation coupler unit from a plurality of available correction-fixation coupler units each including opposing, first and second arms and a body carrying at least one joint mechanism, wherein a number and types of joint mechanisms provided with the selected coupler unit is a function of a number and type of deviations occurring between the first and second vertebrae the coupler unit is intended to align;
connecting the first arm of the first coupler unit to a first anchor fixed to the first vertebra and connecting the second arm of the first coupler unit to a second anchor fixed to the second vertebra;
mobilizing the at least one joint mechanism of the first coupler unit by a calculated amount to a correction arrangement based upon at least one of the determined deviation parameters to manipulate the first and second vertebrae relative to one another and correct the deformity; and
locking the at least one joint mechanism in the correction arrangement to fix the spine in a corrected position.

36. The vertebral system of claim 31, wherein the at least one joint mechanism comprises a driving or input gear and a driven or output gear, and further wherein a ratio of an amount of rotation dialed at the driving gear to an amount of rotation delivered at the driven gear said input-output ratio depends on a number of teeth of the gears, diameter of the gears and a number of starts of the threads of the gears.

37. The vertebral system of claim 31, wherein an amount of rotation of a screwhead or actuator required to operate the at least one joint mechanism to effect a desired vertebral correction in a specific direction is predetermined from calculations of actual vertebral deviation, target vertebral correction, and an input output ratio of the joint mechanism.

38. The vertebral system of claim 31, wherein the at least one joint mechanism is configured to be selectively configured using a robotic system.

39. The vertebral system of claim 31, wherein the at least one joint mechanism is configured to be selectively configured using a torque limiting screwdriver adjusted to generate a torque that is lower than a predetermined threshold torque.

40. The vertebral system of claim 31, wherein the at least one coupler unit is configured to support one or more joint mechanisms, such that collectively the coupler unit is capable of manipulating the vertebra and correcting vertebral deviation in any one or any combination of six degrees of freedom, including sagittal angulation, coronal tilt, axial rotation, medial-lateral translation, anterior-posterior translation, and cranial-caudal translation.

41. The vertebral system of claim 31, wherein the number and types of joint mechanisms within the coupler unit depend on the number and directions of vertebral deviations the coupler unit is intended to correct.

42. The vertebral system of claim 31, wherein the at least one joint mechanism includes a self-locking mechanism comprising at least one of a mechanical locking arrangement, a friction-based locking arrangement, and a singularity locking arrangement.

43. The vertebral system of claim 31, wherein the at least one joint mechanism is configured to be selectively mobilized by at least one of a manual driver, an automated external motor programmable to receive commands from a smart hub, or an internal motor located within the coupler body and controlled by a remote controller.

44. The vertebral system of claim 31, wherein the system, including the at least one correction-fixation coupler unit, is configured to be used for correction and fixation of the spine and to remain implanted in the human body, or to be used for correction of the spine and subsequently removed from the patient after a second fixation apparatus or solid rod is installed to fix the spine in the corrected position.

45. The vertebral system of claim 31, wherein motion of the at least one joint mechanism is calibrated such that a rotation of an actuator or screw head controlling the joint mechanism results in a predictable and calculable amount of either a defined angular rotation or a defined linear displacement at the corresponding end effector arm.

46. The vertebral system of claim 31, wherein at least one joint mechanism is configured to receive operational parameters comprising a specific degree and direction of screw head rotation, to effect a targeted degree and direction of correction of a specific vertebral deviation.

47. The vertebral system of claim 31, wherein the at least one joint mechanism is either a one degree of freedom revolute joint configured to mobilize the first arm and the second arm relative to one another around a single specific rotation axis, or a one degree of freedom translational joint configured to mobilize the first arm and the second arm relative to one another along a single specific linear axis.

48. The vertebral system of claim 31, wherein the at least one joint mechanism includes a locking means configured to provide a plurality of locking conditions including a fully unlocked condition, a self-locking condition and a fully locked condition.

49. The vertebral system of claim 31, wherein the coupler body comprises a first body part carrying the first arm and a second body part carrying the second arm, and further wherein the first body part is connected to the second body part by an articulating joint mechanism configured to enable the first and second body parts to move relative to one another.
